(19) United States
(12) Patent Application Publication
(10) Pub. No.: US 2010/0266528 A1
(43) Pub. Date: Oct. 21, 2010

(54) Systemic Administration of Colony Stimulating Factors to Treat Amyloid Associated Disorders

(75) Inventors: Gordon Wong, Brookline, MA (US); Jane K. Relton, Belmont, MA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc.

(21) Appl. No.: 12/515,374

(22) PCT Filed: Nov. 16, 2007
(86) PCT No.: PCT/US07/24055

§ 371 Date: Mar. 25, 2010

Related U.S. Application Data
(60) Provisional application No. 60/859,483, filed on Nov. 17, 2006.

(51) Int. Cl. A61K 38/19; A61K 31/7088; A61P 25/28; A61P 29/00; A61P 25/16; A61P 3/10; A61P 9/12

(52) U.S. Cl. 424/85.1; 514/44 R

(57) ABSTRACT

The invention relates a method of treating amyloidosis, diseases and disorders associated with amyloid plaque formation, e.g., Alzheimer's disease by increasing tissue resident macrophage activity in an organ or tissue of an animal requiring treatment by systemic administration of a colony stimulating factor. For example, the activity of bone marrow-derived microglial cells in an organ or tissue can be increased by systemic administration of a colony stimulating factor, particularly macrophage colony stimulating factor, either alone or in combination with additional colony stimulating factors, stem cell factors or other compounds capable of treating amyloidosis.

### BACKGROUND OF THE INVENTION

1. Field of the Invention

The invention relates a method of treating diseases and disorders associated with amyloidosis, attributed to the formation and deposition of amyloid plaques by increasing the activity of tissue resident macrophages, e.g., bone marrow derived-microglia, in a tissue or organ of an animal in need of treatment for amyloidosis. Bone marrow derived-microglial cells can reduce the size and quantity of amyloid plaques in an organ or tissue, e.g., β-amyloid plaques in the brain. Amyloid plaques are associated with a variety of diseases and disorders, including Alzheimer's disease (AD). The activity of bone marrow derived-microglial cells in an organ or tissue, including the brain, can be increased by systemic administration of a colony stimulating factor, either alone or in combination with additional colony stimulating factors, stem cell factors or other compounds capable of treating diseases associated with amyloidosis, e.g., Alzheimer's disease.

2. Background

Amyloidosis is a disorder of protein folding (or misfolding) in which soluble proteins form insoluble fibril aggregates that are deposited in extracellular space, progressively disrupting tissue structure and impairing function. Many different unrelated proteins (β-amyloid, immunoglobulin light chains (λ), amyloid A (N-terminal fragment of serum amyloid A), β₂-microglobulin, drusen, wild-type and mutant transthyretin, mutant apolipoproteins, islet amyloid precursor protein, calcitonin, atrial natriuretic protein, huntingtin (intact or poly(Q) rich fragment), human prion protein in “scrapie” form, α-synuclein, tau (wild type or mutant), cystatin C, gelsolin, amylin, lysozyme (mutants), insulin, superoxide dismutase I (wild type or mutants), androgen receptor (intact or poly(Q) rich fragments), ataxins (intact or poly(Q) rich fragments), or TATA box-binding protein (intact or poly(Q) rich fragments)) can form amyloid plaques in vivo, but the fibrils formed by these distinct proteins are remarkably similar in structure. (Dobson, C. M., Protein & Peptide Lett. 13:219-227 (2006)). Amyloid deposits can be systemic or localized. (Meredith, S.C., Ann. N.Y. Acad. Sci. 1066: 181-221, (2005)). In systemic amyloidosis, deposits occur in any organ, except the brain, and can be fatal due to impaired organ function. In localized amyloidosis, the deposits are confined to a particular organ or tissue, including the brain, but often remain clinically silent until later in age, when these localized plaques are associated with onset of serious diseases. Amyloid plaques are observed in conditions which include, but are not limited to: Alzheimer's disease, mild cognitive impairment, mild-to-moderate cognitive impairment, vascular dementia, senile dementia, trisomy 21 (Down's syndrome), hereditary cerebral hemorrhage with amyloidosis of the Dutch-type (HCHWA-D), cerebral amyloid angiopathy (CAA), age-related macular degeneration, multiple myeloma, pulmonary hypertension, congestive heart failure, type II diabetes, rheumatoid arthritis, familial amyloid polyneuropathy (FAP), spongiform encephlaopathies, Parkinson's disease, primary systemic amyloidosis, secondary systemic amyloidosis, fronto-temporal dementias, senile systemic amyloidosis, hereditary cerebral amyloid angiopathy, haemodialysis-related amyloidosis, familial amyloid polyneuropathy III, Finnish hereditary systemic amyloidosis, medullary carcinoma of the thyroid, atrial amyloidosis, hereditary non-neuropathic systemic amyloidosis, injection-localized amyloidosis, hereditary renal amyloidosis, amyotrophic lateral sclerosis, Huntington's disease, spinal and bulbar muscular atrophy, spinocerebellar ataxias and spinocerebellar ataxia 17. (Dobson, C. M., Protein & Peptide Lett. 13:219-227 (2006)).

Characteristic features of localized amyloidosis disorder is observed in Alzheimer's disease (AD), which is a neurodegenerative disorder that results in progressive loss of memory, cognition, reasoning, judgment and emotional stability and ultimately death. A pathologic hallmark of AD is the presence of amyloid plaques in the brain. The major constituent of amyloid plaques associated with AD is Aβ peptide, which is derived proteolytically from Amyloid Precursor Protein (APP) by β-secretase (RACE) and γ-secretase (Presenilin-1,2 and associated proteins). APP also is converted to innocuous peptides and protein fragments by α-secretases and γ-secretase. Genetic studies of human familial AD (FAD) have found that mutations in APP and/or Presenilins alter the production of total Aβ peptide or the ratio of fibrillogenic Aβ42-3 peptide to other APP cleavage products. In addition, mice that express mutant human FAD versions of APP with or without mutant presenilins exhibit amyloid plaque deposition and cognitive impairment.

Microglial cells are physically associated and integrated into amyloid plaques and thereby implicated in the pathophysiology of amyloidosis, e.g., AD. In familial amyloid polyneuropathy (FAP), pro-inflammatory cytokines, such as TNFα, IL-1β and macrophage-colony stimulating factor (M-CSF) were upregulated, especially in the endoneurial axons. (Sousa, M. M. and Saraiva M. J. Prog. In Neurobiol. 71:385-400 (2003)). This observed increase in cytokines in FAP patients was associated with the later stages of the disorder. Increased M-CSF levels have also been reported in primary amyloidosis (AL), secondary amyloidosis, and systemic amyloidosis. (Rysava et al., Biochem. And Molec. Biol. Int. 47(5):845-850, (1999)). In brain, M-CSF is also physically associated with β-amyloid plaques and potentially influences the pathophysiology of AD. However, it remains a question whether altering the status of microglial cells and/or M-CSF activity within a specific organ or tissue, e.g., the brain, would affect the course of systemic or localized amyloidosis, e.g., AD.

Microglial cells are derived from monocytic progenitors that are responsive to macrophage colony stimulating factor (M-CSF). Despite its safe toxicity profile and its ability to incite an overt monocytosis, M-CSF was unable to show sufficient clinical efficacy in oncology models tested. There is evidence to suggest, however, that M-CSF may affect the cell biology of tissue resident macrophages, including microglia, in an organ or tissue, including the brain, and thereby affect the course of amyloid disorders, e.g., AD. M-CSF is a potential treatment for amyloidosis, including AD.

M-CSF is a tissue resident macrophage growth factor in vitro and has been found to promote both anti-inflammatory and pro-inflammatory functions. For example, M-CSF has been shown in vitro to increase the ability of microglial cells to phagocytose β-amyloid plaques. (Mitrasinovic et al., Neur. Letters 344: 185-188 (2003)). Additional experiments indicate that transfection of the M-CSF receptor into microglial cells results in an increase in microglial cells functionality, i.e., the ability to phagocytose β-amyloid plaque. (Mitrosinovic & Murphy, J. Biol. Chem. 277:29889-29896 (2002) and Mitrosinovic & Murphy, Neurobiol. Aging 24:807-815 (2003)).

Tissue resident macrophages, including microglial cells, appear to attenuate β-amyloid plaque formation processes through phagocytosis or through the focal release of metalloproteases. They may also incite the neurodegenerative process by processing amyloid plaques into toxins thereby inciting an inflammatory response. These responses may be temporally dependent on the stages of amyloid plaque formation. (Maim et al., Neurobiol. Dis. 18:134-42 (2005)).

The brain has both resident and bone marrow-derived microglial cells. The latter arise from monocytic hematopoietic progenitors. Recent work by Simard et al. (Neuron 49:489-502 (2006)) has shown that selectively depleting bone marrow derived microglial cells leads to both an increase in amyloid plaque size and numbers in the APP/PS1 murine model of AD. However, the converse hypothesis of whether increasing bone marrow-derived microglial cells or enhancing bone marrow-derived microglial cells function (either pro or anti inflammatory responses) leads to a decrease in amyloid plaque size and formation has not been proposed nor tested to date.

There is good experimental evidence from labeled hematopoietic stem cells that a peripheral monocytosis may result in brain microglial cells re-population. (Malm et al., Neurobiol. Dis. 18:134-42 (2005) and Simard et al. (Neuron 49:489-502 (2006)). Hematopoietic colony stimulating factors (CSF) have been shown clinically to enhance the rate of bone marrow engraftment and repopulation. CSFs have also been used clinically in humans to increase erythroid, myeloid and lymphoid cell numbers, survival and function.

M-CSF will augment in vitro microglial cell production of pro-inflammatory factors Il-1, IL-6 and nitric oxide. (Murphy et al., J. Biol. Chem. 273:20967-71 (1998)) and when coupled with beta amyloid induces microglial cells mediated neurotoxicity. (Li et al., J. Neurochem. 91:623-633 (2004)). Which effects would predominate in vivo has yet to be explored.

M-CSF receptor and M-CSF are upregulated in the brains of AD mice (AβPP V717F). (Mitrosinovic et al., J. Biol. Chem. 276:30142-9 (2001), Murphy et al., Am. J. Pathol. 157:895-904 (2000)). A naturally occurring deletion in the M-CSF gene in mice, the osteoporotic mouse (op-/op-)(Marks & Lane, Journal of Heredity 67:11-18 (1976)), leads to a decrease in monocytes, macrophages, brain microglial cells, and osteoclasts. In the osteoporotic mouse where there is no M-CSF, there are reports of an increase in amyloid plaque and reduced brain microglial cells. (Sasaki et al. Neuro. 20:134-42 (2000), Kaku et al., Brain. Res. Protoc. 12:104-108 (2003)). Kawata et al. (J. Int. Med. Res. 33:654-60 (2006)) has shown that a single injection of M-CSF directly into the brain is able increase the number of microglial cells observed and to decrease the rate and size of plaque formation. However there has been no proposal or evidence to date that systemic administration of M-CSF affects the activity of bone marrow-derived microglial cells in the brain.

Granulocyte colony stimulating factor (G-CSF) is a hematopoietic growth factor named for its role in the proliferation and differentiation of myeloic lineage. Administration of G-CSF mobilized hematopoietic stem cells (HSCs) from the bone marrow into the peripheral blood. (Bodine, D. M. et. al., Blood 84:1482-1491 (1994)). Tsai et al. (Tsai, K-J. et al., J. Exp. Biol., DOI:10.10184/jem.20062481; E pub ahead of print (2007)) found that G-CSF induced stem cell release from the bone marrow, stimulated neurogenesis surrounding the Aβ plaques in mouse brain, and substantially improved the neurological function of AD mice. However there has been no proposal or evidence to date that systemic administration of G-CSF affects the activity of bone marrow-derived microglial cells in the brain.

M-CSF receptor is upregulated in brain microglial cells of AD, FAP and ALS patients. (Sousa, M. M and Saraiva, M. J., Progress in Neurobiology 71(5):386-397 (2003) and Akiyama et al., Brain Res. 639:171-174 (1994)). AD patients' brains have shown increased neuronal expression of M-CSF in the proximity of amyloid plaques and microglial cells. One study found that while the concentration of M-CSF in the cerebro-spinal fluid of Alzheimer's patients is 5 fold increased compared with age matched controls, there is no detectable increase or disease correlation with serum M-CSF antigen levels. (Yan et al., PNAS 94:5296-301 (1997)). In contrast, a recent study found a decrease in M-CSF in the plasma of AD patients. (Wyss-Coray et al., Classification and prediction of clinical Alzheimer's diagnosis based on plasma signaling proteins, Nat. Med, October 14, epub ahead of print (2007)).

The evidence suggests that there is some role for M-CSF or G-CSF in the regulation of macrophage function throughout the body. There is a need in the art for additional methods to treat amyloid related diseases or disorders. Tissue resident macrophages in affected organs and tissues, e.g., microglia in the brain, are an important therapeutic target.

### BRIEF SUMMARY OF THE INVENTION

The present invention is based on the discovery that systemic (e.g., not intracranial) administration of a growth factor, for example, a colony stimulating factor, for example, M-CSF, reduces amyloid plaque deposition, e.g., β-amyloid plaques, and amyloid polypeptide, e.g., β-amyloid, aggregation. Based on this discovery, the invention features methods of treating disorders associated with the deposition of amyloid plaques, including AD, by the systemic administration of M-CSF or G-CSF, alone or in combination with other CSFs, stem cell factors or therapeutic agents effective to treat amyloidosis, e.g., AD.

In some embodiments, the invention provides a method of increasing tissue resident macrophage, including bone marrow-derived microglial, activity in amyloidosis affected organs or tissues, e.g., the brain, of an animal, comprising systemically administering to an animal a composition comprising (a) an isolated colony stimulating factor polypeptide or active variant, fragment or derivative thereof; (b) an isolated polynucleotide encoding a colony stimulating factor polypeptide or active variant, fragment or derivative thereof, through operable association with a promoter; or (c) a combination of (a) and (b). The composition is administered in an amount effective to increase tissue resident macrophage activity, including bone marrow-derived microglial cell activity, in an organ or tissue, e.g., the brain.

In some embodiments, the organ or tissue is the central nervous system (CNS: brain and spinal cord), peripheral nervous system (PNS: nerve trunks, plexuses, sensory and autonomic ganglia), liver, spleen, pancreas, kidney, stomach, heart, gastrointestinal tract, thyroid, lung, salivary glands, cerebral blood vessels or general blood vessels of an animal afflicted with amyloidosis. In some embodiments, said organ is the brain of an animal afflicted with AD.

In some embodiments, the invention provides a method for reducing amyloid polypeptide or amyloid deposition or plaques in an animal, comprising systemically administering a therapeutically effective amount of a growth factor polypeptide, for example, a colony stimulating factor, for example, M-CSF to the animal. In some embodiments, amyloid plaques are present in association with a disorder. In some embodiments, the disorder is AD, mild cognitive impairment, mild-to-moderate cognitive impairment, vascular dementia, senile dementia, trisomy 21 (Down's syndrome), hereditary cerebral hemorrhage with amyloidosis of the Dutch-type (HCHWA-D), cerebral amyloid angiopathy (CAA), age related macular degeneration, multiple myeloma, pulmonary hypertension, congestive heart failure, type II diabetes, rheumatoid arthritis, familial amyloid polyneuropathy (FAP), spongiform encephalopathies, Parkinson's disease, primary systemic amyloidosis, secondary systemic amyloidosis, fronto-temporal dementias, senile systemic amyloidosis, hereditary cerebral amyloid angiopathy, haemodialysis-related amyloidosis, familial amyloid polyneuropathy III, Finnish hereditary systemic amyloidosis, medullary carcinoma of the thyroid, atrial amyloidosis, hereditary non-neuropathic systemic amyloidosis, injection-localized amyloidosis, hereditary renal amyloidosis, amyotrophic lateral sclerosis (ALS), Huntington's disease, spinal and bulbar muscular atrophy, and spinocerebellar ataxia.

In some embodiments, the invention provides a method for reducing amyloid polypeptide or amyloid deposition or plaques in an animal, comprising systemically administering a therapeutically effective amount of a growth factor polypeptide, for example, a colony stimulating factor, for example, M-CSF to the animal. In some embodiments, amyloid plaques comprise a protein selected from a group consisting of β-amyloid, immunoglobulin light chain (type AL amyloidosis), serum amyloid A (type AA amyloidosis), β₂-microglobulin (type Aβ₂M amyloidosis), drusen, wild-type or mutant transthyretin (type ATTR amyloidosis), mutant apolipoprotein AI (type AApoAI amyloidosis), mutant apolipoprotein All (type AApoAII amyloidosis), islet amyloid precursor protein, calcitonin, atrial natriuretic protein, huntingtin (intact or poly(Q) rich fragment), human prion protein in “scrapie” form, α-synuclein, tau (wild type or mutant), cystatin C (type ACys amyloidosis), gelsolin (type AGel amyloidosis), amylin, mutant lysozyme (type ALys amyloidosis), insulin, superoxide dismutase I (wild type or mutants), androgen receptor (intact or poly(Q) rich fragments), ataxins (intact or poly(Q) rich fragments), TATA box-binding protein (intact or poly(Q) rich fragments), mutant fibrinogen A α-chain (type AFib amyloidosis), β-protein precursor (type Aβ amyloidosis) and a combination of amyloid proteins.

In some embodiments, the invention provides a method of preventing or treating a disorder associated with amyloid polypeptide or amyloid deposition or plaques, comprising systemically administering an amount of a growth factor, for example, a colony stimulating factor, for example, M-CSF, effective to increase the activity of bone marrow-derived microglial cells in the amyloidosis-afflicted organs or tissues of said animal.

In some embodiments, the amyloid aggregates or plaques are phagocytosed by said bone marrow-derived microglial cells. In some embodiments, the phagocytosis of the amyloid plaques or aggregates observed in an amyloidosis-afflicted organ or tissue, e.g., the brain, results in a reduction in the size of the amyloid plaques in said organ or tissue, including the brain. In some embodiments, the phagocytosis of the amyloid plaques results in a reduction in the number of the amyloid plaques.

In some embodiments, the invention provides a method of preventing or treating a disorder associated with β-amyloid polypeptide or β-amyloid deposition or plaques, comprising systemically administering an amount of a growth factor, for example, a colony stimulating factor, for example, M-CSF, effective to increase the activity of bone marrow-derived microglial cells in the brain of the said animal. In some embodiments, the disorder is AD.

In some embodiments, the β-amyloid plaques are phagocytosed by said bone marrow-derived microglial cells. In some embodiments, the phagocytosis of the β-amyloid associated with AD results in a reduction in the size of the β-amyloid plaques in brain. In some embodiments, the phagocytosis of the β-amyloid plaques results in a reduction in the number of the β-amyloid plaques.

In some embodiments, the growth factor, for example, a colony stimulating factor, for example, M-CSF, is administered alone. In some embodiments, the growth factor, for example, a colony stimulating factor, for example, M-CSF, is administered in association with a therapeutic agent effective to treat, prevent or ameliorate a disorder associated with amyloid plaques, for example, amyloidosis. In some embodiments, the growth factor, for example, a colony stimulating factor, for example, M-CSF, is administered in association with a stem cell factor, for example, G-CSF, IL-3, IL-5, IL-6, IL-11, or kit ligand. In some embodiments, the growth factor, for example, a colony stimulating factor, for example, M-CSF, may be concurrently administered intracranially.

In some embodiments, the growth factor, for example, a colony stimulating factor, for example, M-CSF, is administered alone. In some embodiments, the growth factor, for example, a colony stimulating factor, for example, M-CSF, is administered in association with a therapeutic agent effective to treat, prevent or ameliorate a disorder associated with β-amyloid plaques, including AD. In some embodiments, the growth factor, for example, a colony stimulating factor, for example, M-CSF, is administered in association with a stem cell factor, for example, granulocyte colony stimulating factor, IL-3, IL-5, IL-6, IL-11, or kit ligand. In some embodiments, the growth factor, for example, a colony stimulating factor, for example, M-CSF, may be concurrently administered intracranially.

In some embodiments, a growth factor polypeptide fragment is administered. In some embodiments, a colony stimulating factor polypeptide fragment is administered. In some embodiments, an M-CSF polypeptide fragment is administered.

In some embodiments, the invention provides a method of increasing bone, marrow-derived microglial cell activity in an organ or tissue of an animal, comprising systemically administering an isolated polynucleotide encoding a colony stimulating factor polypeptide, or a fragment thereof, through operable association with a promoter. In some embodiments, the polynucleotide is delivered via an expression vector, for example, a viral vector.

In some embodiments, the invention provides a method of increasing bone marrow-derived microglial cell activity in the brain of an animal, comprising systemically administering an isolated polynucleotide encoding a colony stimulating factor polypeptide, or a fragment thereof, through operable association with a promoter. In some embodiments, the polynucleotide is delivered via an expression vector, for example, a viral vector.

In some embodiments, the growth factor for example, a colony stimulating factor, for example, M-CSF further comprises a fusion moiety. In some embodiments, the fusion moiety is an immunoglobulin moiety. In some embodiments, the immunoglobulin moiety is an Fc moiety. In other embodiments, the fusion moiety is a serum albumin moiety, a targeting moiety, a reporter moiety, a purification-facilitating moiety, and a combination of two or more thereof.

In some embodiments, the growth factor, for example, a colony stimulating factor, for example, M-CSF is administered once per day, continuously, intermittently, before, during or after formation of amyloid plaques, e.g., β-amyloid plaques, or amyloid aggregates, e.g., β-amyloid aggregates, until there is a reduction in the size and/or formation of amyloid plaques, e.g., β-amyloid plaques or amyloid aggregates, e.g., β-amyloid aggregates, or a combination of two or more thereof.

In some embodiments, the therapeutically effective amount is from between about 50 and about 100 μg/kg body weight per day. In some embodiments, the therapeutically effective amount is from between about 60 and about 100 μg/kg body weight per day. In some embodiments, the therapeutically effective amount is from between about 70 and about 100 μg/kg body weight per day. In some embodiments, the therapeutically effective amount is from between about 80 and about 100 μg/kg body weight per day. In some embodiments, the therapeutically effective amount is from between about 90 and about 100 μg/kg body weight per day. In some embodiments, the therapeutically effective amount is from between about 50 and about 60 μg/kg body weight per day. In some embodiments, the therapeutically effective amount is from between about 50 and about 70 μg/kg body weight per day. In some embodiments, the therapeutically effective amount is from between about 50 and about 80 μg/kg body weight per day. In some embodiments, the therapeutically effective amount is from between about 50 and about 90 μg/kg body weight per day. In some embodiments, the therapeutically effective amount is from between about 60 and about 70 μg/kg body weight per day. In some embodiments, the therapeutically effective amount is from between about 60 and about 80 μg/kg body weight per day. In some embodiments, the therapeutically effective amount is from between about 60 and about 90 μg/kg body weight per day. In some embodiments, the therapeutically effective amount is from between about 70 and about 80 μg/kg body weight per day. In some embodiments, the therapeutically effective amount is from between about 70 and about 90 μg/kg body weight per day. In some embodiments, the therapeutically effective amount is from between about 50 and about 200 μg/kg body weight per day. In some embodiments, the therapeutically effective amount is from between about 1 and about 500 μg/kg body weight per day.

In some embodiments, the growth factor, for example, a colony stimulating factor, for example, M-CSF, composition administered to the animal further comprises a pharmaceutically acceptable excipient.

In some embodiments, the animal is a vertebrate. In some embodiments, the animal is a mammal. In some embodiments, the animal is a human.

### BRIEF DESCRIPTION OF THE DRAWING

FIG. 1 depicts the effect of 10 weeks of systemic treatment with M-CSF in improving memory of Alzheimer's disease model APP⁺ mice.

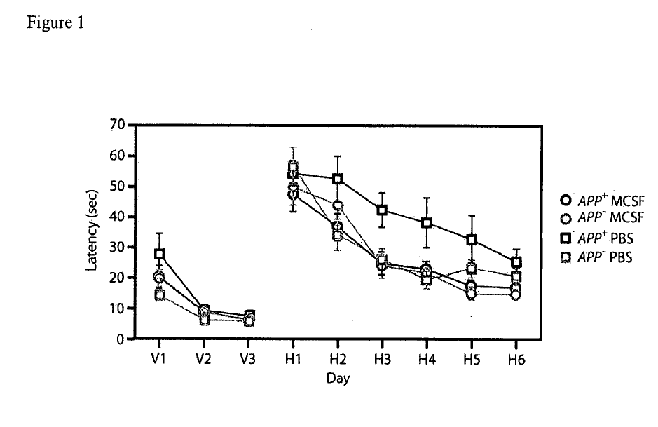

Specifically, FIG. 1 depicts performance of mice in the latency test with either a Visual (V1-V3) or a Hidden platform (H1-H6) over a period of 3 and 6 days respectively. APP⁺, M-CSF mice are denoted with black circles; APP⁻, M-CSF mice are denoted with grey circles; APP⁺, PBS mice are denoted with black squares; and APT, PBS mice are denoted with grey squares.

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

In order to further define this invention, the following terms and definitions are provided.

It is to be noted that the term “a” or “an” entity, refers to one or more of that entity; for example, “a polypeptide,” is understood to represent one or more polypeptides. As such, the terms “a” (or “an”), “one or more,” and “at least one” can be used interchangeably herein.

Throughout this specification and claims, the word “comprise,” or variations such as “comprises” or “comprising,” indicate the inclusion of any recited integer or group of integers but not the exclusion of any other integer or group of integers.

As used herein, the term “consists of,” or variations such as “consist of” or “consisting of,” as used throughout the specification and claims, indicate the inclusion of any recited integer or group of integers, but that no additional integer or group of integers may be added to the specified method, structure or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, “antibody” means an intact immunoglobulin, or an antigen-binding fragment thereof. Antibodies of this invention can be of any isotype or class (e.g., M, D, G, E and A) or any subclass (e.g., G1-4, A1-2) and can have either a kappa (κ) or lambda (λ) light chain.

As used herein, “Fc” means a portion of an immunoglobulin heavy chain that comprises one or more heavy chain constant region domains, CH1, CH2 and CH3. For example, a portion of the heavy chain constant region of an antibody that is obtainable by papain digestion.

As used herein, “humanized antibody” means an antibody in which at least a portion of the non-human sequences are replaced with human sequences. Examples of how to make humanized antibodies may be found in U.S. Pat. Nos. 6,054,297, 5,886,152 and 5,877,293.

As used herein, “chimeric antibody” means an antibody that contains one or more regions from a first antibody and one or more regions from at least one other antibody. The first antibody and the additional antibodies can be from the same or different species.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been identified and separated, fractionated, or partially or substantially purified by any suitable technique.

In the present invention, a “polypeptide fragment” refers to a short amino acid sequence of a larger polypeptide. Protein fragments may be “free-standing,” or comprised within a larger polypeptide of which the fragment forms a part of region. Representative examples of polypeptide fragments of the invention, include, for example, fragments comprising about 5 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, about 100, about 200, and about 500 amino acids or more in length.

The terms “fragment,” “variant,” and “derivative” when referring to a polypeptide of the present invention include any polypeptide which retains at least some biological activity. Polypeptides as described herein may include fragment, variant, or derivative molecules without limitation, so long as the polypeptide still serves its function. Growth factor, colony stimulating factor, or M-CSF polypeptides and polypeptide fragments of the present invention may include proteolytic fragments, deletion fragments and in particular, fragments which more easily reach the site of action when delivered to an animal. Polypeptide fragments further include any portion of the polypeptide which comprises an antigenic or immunogenic epitope of the native polypeptide, including linear as well as three-dimensional epitopes. Growth factor, colony stimulating factor or M-CSF polypeptides and polypeptide fragments of the present invention may comprise variant regions, including fragments as described above, and also polypeptides with altered amino acid sequences due to amino acid substitutions, deletions, or insertions. Variants may occur naturally, such as an allelic variant. By an “allelic variant” is intended alternate forms of a gene occupying a given locus on a chromosome of an organism. Genes II, Lewin, B., ed., John Wiley & Sons, New York (1985). Non-naturally occurring variants may be produced using art-known mutagenesis techniques. Growth factor, colony stimulating factor or M-CSF polypeptides and polypeptide fragments of the invention may comprise conservative or non-conservative amino acid substitutions, deletions or additions. Growth factor, colony stimulating factor or M-CSF polypeptides and polypeptide fragments of the present invention may also include derivative molecules. As used herein a “derivative” of a polypeptide or a polypeptide fragment refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

As used herein the term “disulfide bond” includes the covalent bond formed between two sulfur atoms. The amino acid cysteine comprises a thiol group that can form a disulfide bond or bridge with a second thiol group.

As used herein, “fusion protein” means a protein comprising a first polypeptide linearly connected, via peptide bonds, to a second, polypeptide. The first polypeptide and the second polypeptide may be identical or different, and they may be directly connected, or connected via a peptide linker (see below).

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can contain the nucleotide sequence of the full length cDNA sequence, including the untranslated 5′ and 3′ sequences, the coding sequences. A polynucleotide may comprise a conventional phosphodiester bond or a non-conventional bond (e.g., an amide bond, such as found in peptide nucleic acids (PNA)). The polynucleotide can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotides can be composed of triple-stranded regions comprising RNA or DNA or both RNA and DNA. polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a growth factor, colony stimulating factor or M-CSF polypeptide or polypeptide fragment of the invention contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, a polynucleotide or nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding a growth factor, colony stimulating factor or M-CSF polypeptide or polypeptide fragment of the present invention. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is RNA, for example, in the form of messenger RNA (mRNA).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

A “linker” sequence is a series of one or more amino acids separating two polypeptide coding regions in a fusion protein. A typical linker comprises at least 5 amino acids. Additional linkers comprise at least 10 or at least 15 amino acids. In certain embodiments, the amino acids of a peptide linker are selected so that the linker is hydrophilic. The linker (Gly-Gly-Gly-Gly-Ser)₃ (SEQ ID NO: 7) is a preferred linker that is widely applicable to many antibodies as it provides sufficient flexibility. Other linkers include Glu Ser Gly Arg Ser Gly Gly Gly Gly Ser Gly Gly Gly Gly Ser (SEQ ID NO: 8), Glu Gly Lys Ser Ser Gly Ser Gly Ser Glu Ser Lys Ser Thr (SEQ ID NO: 9), Glu Gly Lys Ser Ser Gly Ser Gly Ser Glu Ser Lys Ser Thr Gln (SEQ ID NO: 10), Glu Gly Lys Ser Ser Gly Ser Gly Ser Glu Ser Lys Val Asp (SEQ ID NO: 11), Gly Ser Thr Ser Gly Ser Gly Lys Ser Ser Glu Gly Lys Gly (SEQ ID NO: 12), Lys Glu Ser Gly Ser Val Ser Ser Glu Gln Leu Ala Gln Phe Arg Ser Leu Asp (SEQ ID NO: 13), and Glu Ser Gly Ser Val Ser Ser Glu Glu Leu Ala Phe Arg Ser Leu Asp (SEQ ID NO: 14). Examples of shorter linkers include fragments of the above linkers, and examples of longer linkers include combinations of the linkers above, combinations of fragments of the linkers above, and combinations of the linkers above with fragments of the linkers above.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product, and the translation of such mRNA into polypeptide(s), as well as any processes which regulate either transcription or translation. If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “treat” or “treatment” refer to both therapeutic treatment and prophylactic or preventative measures, wherein the object is to prevent or slow down (lessen) an undesired physiological change or disorder, such as the progression of AD. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

As used herein, phrases such as “a subject that would benefit from systemic administration of growth factor, for example, an M-CSF polypeptide or polypeptide fragment of the present invention” and “an animal in need of treatment” includes subjects, such as mammalian subjects, that would benefit from systemic administration of a growth factor, for example, a colony stimulating factor, for example, an M-CSF polypeptide or polypeptide fragment of the present invention used, e.g., for detection (e.g., for a diagnostic procedure) and/or for treatment, i.e., palliation or prevention of a disease such as AD, with a growth factor, colony stimulating factor or M-CSF polypeptide or polypeptide fragment of the present invention. As described in more detail herein, the polypeptide or polypeptide fragment can be used in unconjugated form or can be conjugated, e.g., to a drug, prodrug, or an isotope.

As used herein, a “therapeutically effective amount” or “an amount effective” refers to an amount effective, at dosages and for periods of time necessary, to achieve the desired therapeutic result. A therapeutic result may be, e.g., lessening of symptoms, prolonged survival, improved mobility, and the like. A therapeutic result need not be a “cure.” A therapeutic result may also be prophylactic. Typically, since a prophylactic dose is used in subjects prior to or at an earlier stage of disease, the prophylactically effective amount will be less than the therapeutically effective amount.

As used herein, “bone marrow-derived microglial cell activity” refers to any increase in activity of bone marrow-derived microglial cells. The increase in activity can be effected by, for example, an increase in the number of bone marrow-derived microglial cells, an increase in the activation of bone marrow-derived microglial cells, an increase in targeting of bone marrow-derived microglial cells, or a combination of two or more thereof.

As used herein, “systemic administration” means any form of administration other than intracranially. Examples of systemic administration include, but are not limited to, oral administration, nasal administration, parenteral administration, transdermal administration, topical administration, intraocular administration, intrabronchial administration, intraperitoneal administration, intravenous administration, subcutaneous administration, intramuscular administration, buccal administration, sublingual administration, vaginal administration, intraheptic, intracardiac, intrapancreatic, transplantation, by inhalation, by an implanted pump, or a combination of two or more thereof.

The invention is directed to a method of treating an animal by systemically administering to an animal in need of such treatment, growth factors, for example, colony stimulating factors (CSFs) or active fragments, derivatives or variants thereof effective to increase the activity of tissue resident macrophages in an organ or tissue of an animal. For example, the present invention provides growth factors, for example, CSF polypeptides and polypeptide fragments, which result in an increase in the presence, activation and/or targeting of bone marrow derived-microglial cells in an organ or tissue, such as the brain. Bone marrow derived-microglial cells can reduce the size and quantity of amyloid plaques in an organ or tissue, such as the reduction in size and quantity of β-amyloid plaques in the brain. Amyloid plaques and vascular amyloid deposits (amyloid angiopathy) are present in, for example, in AD, mild cognitive impairment, mild-to-moderate cognitive impairment, vascular dementia, senile dementia, trisomy 21 (Down's syndrome), hereditary cerebral hemorrhage with amyloidosis of the Dutch-type (HCHWA-D), cerebral amyloid angiopathy (CAA) and AD. Amyloid plaques are also present in age-related macular degeneration, multiple myeloma, pulmonary hypertension, congestive heart failure, type II diabetes, rheumatoid arthritis, familial amyloid polyneuropathy (FAP), spongiform encephlaopathies, Parkinson's disease, primary systemic amyloidosis, secondary systemic amyloidosis, fronto-temporal dementias, senile systemic amyloidosis, hereditary cerebral amyloid angiopathy, haemodialysis-related amyloidosis, familial amyloid polyneuropathy III, Finnish hereditary systemic amyloidosis, medullary carcinoma of the thyroid, atrial amyloidosis, hereditary non-neuropathic systemic amyloidosis, injection-localized amyloidosis, hereditary renal amyloidosis, amyotrophic lateral sclerosis, Huntington's disease, spinal and bulbar muscular atrophy, spinocerebellar ataxias and spinocerebellar ataxia 17 and inclusion myositis. As such, systemic administration of CSF polypeptides or polypeptide fragments is effective to treat amyloid plaque related diseases or disorders, e.g., AD. The growth factors, for example, CSF polypeptides or fragments, can be administered alone or in combination with stem cell factors and/or therapeutic agents effective to treat amyloid related diseases or disorders, e.g., β-amyloid related diseases or disorders, e.g., AD.

The invention is also directed to a method of treating an animal by systemically administering to an animal in need of such treatment certain growth factors, colony stimulating factor or M-CSF polypeptides or polypeptide fragments effective to increase the activity of tissue resident macrophages, for example, bone marrow-derived-microglial cells in the brain of an animal. For example, the present invention provides growth factors, colony stimulating factors or M-CSF polypeptides and polypeptide fragments which result in an increase in the presence, activity and/or targeting of bone marrow derived-microglial cells in the brain. As such, systemic administration of growth factors, colony stimulating factors or M-CSF polypeptides or polypeptide fragments is effective to treat β-amyloid plaque related diseases or disorders, e.g., AD. The growth factors, colony stimulating factors or M-CSF polypeptides or fragments can be administered alone or in combination with other CSFs, stem cell factors and/or therapeutic agents effective to treat β-amyloid plaque related diseases or disorders, e.g., AD.

Growth Factor Polypeptides and Polypeptide Fragments

The present invention is directed to the systemic administration of certain growth factor polypeptides or fragments, variants or derivatives thereof, e.g., colony stimulating factors (CSFs), granulocyte CSF, granulocyte-macrophage CSF or M-CSF, for treating or preventing disorders associated with amyloid polypeptide or amyloid deposition or plaques, for example, amyloidosis. The present invention is also directed to the systemic administration of an identified, isolated polynucleotide encoding a colony stimulating factor polypeptide or active variant, fragment or derivative thereof. The polynucleotide can be delivered via an expression vector, e.g., a viral vector.

The present invention is directed to the systemic administration of certain growth factor polypeptides or fragments, variants or derivatives thereof, e.g., colony stimulating factors (CSFs), granulocyte CSF, granulocyte-macrophage CSF or M-CSF, for treating or preventing disorders associated with β-amyloid polypeptide or β-amyloid deposition or plaques, for example, AD. The present invention is also directed to the systemic administration of an identified, isolated polynucleotide encoding a colony stimulating factor polypeptide or active variant, fragment or derivative thereof. The polynucleotide can be delivered via an expression vector, e.g., a viral vector.

The present invention is also directed to the systemic administration of growth factors, for example, CSFs or active variants, fragments or derivatives thereof which have a second polypeptide fused to the growth factor, for example, CSF for treating or preventing disorders associated with amyloid plaques, e.g., amyloidosis. Examples of the second polypeptide include, but are not limited an immunoglobulin Fc region, a serum albumin moiety, a targeting moiety, a reporter moiety, a purification-facilitating moiety, or a combination of two or more second polypeptides.

The present invention is also directed to the systemic administration of growth factors, for example, CSFs or active variants, fragments or derivatives thereof which have a second polypeptide fused to the growth factor, for example, CSF for treating or preventing disorders associated with β-amyloid plaques, e.g., AD. Examples of the second polypeptide include, but are not limited an immunoglobulin Fc region, a serum albumin moiety, a targeting moiety, a reporter moiety, a purification-facilitating moiety, or a combination of two or more second polypeptides.

The systemic administration of growth factors or CSFs for treating or preventing disorders associated with amyloid polypeptide or amyloid deposition or plaques, e.g., AD can also occur in combination with a stem cell factor or active variant, fragment or derivative thereof, e.g., granulocyte colony stimulating factor, IL-3, IL-5, IL-6, IL-11, kit ligand or a combination of two or more thereof. In addition the growth factors or CSFs can be co-administered with a pharmaceutical compound effective for treating, preventing or inhibiting amyloid polypeptide or an amyloid related disorder or disease, e.g., AD.

Hematopoietic CSFs may be applied to increase the concentration of microglial cells and enhance their functional biology in an organ or tissue, e.g., the brain. For example, such microglial cells may attenuate the rate of amyloid plaque formation in a brain afflicted with AD, eliminate plaques through phagocytosis, reduce plaque derived neurotoxins and improve brain and cognitive functions.

The monocytic origins of microglial cells would point to colony stimulating factors such as M-CSF or granulocyte macrophage (GM) CSF used singly or in combination with stem cell factors such as granulocyte (G) CSF, IL-3, and kit ligand. Of the candidate colony stimulating factors, M-CSF is the most promising first choice. M-CSF is a growth factor for monocytes and macrophages. To date, the systemic administration of such factors has yet to be empirically demonstrated to either increase bone marrow-derived microglial cells concentrations in amyloidosis disease models or to treat disease pathophysiology.

M-CSF is 70- to 90-kD membrane bound disulfide-linked homodimer glycoprotein that stimulates proliferation and supports survival and differentiation of cells of the mononuclear phagocyte series. Pandit et al, Science 258: 1358-62 (1992). Three related cDNA clones of human M-CSF have been isolated, representing long (beta) intermediate (gamma) and short (alpha) splicing variants from the single M-CSF gene. Cerritti, D. P. et al. Mol. Immunol. 25: 761-70 (1988). The isoforms are shown below as SEQ ID NOS. 1, 2 and 3, respectively.

Deletion studies on recombinant M-CSF have shown that in vitro activity is fully retained in analogs consisting of the first 150 amino acids of the mature M-CSF. This region is common to all three splice variants and contains a unique four-helical structure that defines a family of hormones with which M-CSF shares little significant amino acid identity. This family includes human growth hormone, GM-CSF, G-CSF, M-2, IL-4, IL-5, leukemia inhibitory factory, and likely IL-3 and IL-6. Cerritti, D. P. et al. Mol. Immunol. 25: 761-70 (1988).

The active receptor binding moiety found in M-CSF is a covalently linked dimer. The dimer is formed by linking two of the unique four-helical structures together to form a flat elongated structure. Mutation experiments have shown that the first 6 cysteine residues in each helical chain are essential for biological activity. Pandit et al, Science 258: 1358-62 (1992).

M-CSF is rich in proline residues (63/544 residues) mostly found in the C-terminal 377 residues. The proline-rich composition of the C-terminal region might be involved in a step of post-translational modification such as C-terminal processing or glycosylation. C-terminally truncated M-CSF, lacks the membrane spanning domain and is secreted. However, the secreted M-CSF retains activity indicating that at least the 377 C-terminal amino acids residue are dispensable and the M-CSF molecule does not require the C-terminal region in order to fold into its active dimeric form. Takahashi et al., Biochem. Biophys. Res. Comm 161(2): 892-901 (1989).

The human M-CSF long (beta) intermediate (gamma) and short (alpha) splicing variants are shown below as SEQ ID NOS. 1, 2 and 3, respectively.

Full-Length Human M-CSF (SEQ ID NO: 1):

MTAPGAAGRCPPTTWLGSLLLLVCLLASRSITEEVSEYCSHMIGSGHLQS
LQRLIDSQMETSCQITFEFVDQEQLKDPVCYLKKAFLLVQDIMEDTMRFR
DNTPNAIAIVQLQELSLRLKSCFTKDYEEHDKACVRTFYETPLQLLEKVK
NVFNETKNLLDKDWNIFSKNCNNSFAECSSQDVVTKPDCNCLYPKAIPSS
DPASVSPHQPLAPSMAPVAGLTWEDSEGTEGSSLLPGEQPLHTVDPGSAK
QRPPRSTCQSFEPPETPVVKDSTIGGSPQPRPSVGAFNPGMEDILDSAMG
TNWVPEEASGEASEIPVPQGTELSPSRPGGGSMQTEPARPSNFLSASSPL
PASAKGQQPADVTGTALPRVGPVRPTGQDWNHTPQKTDHPSALLRDPPEP
GSPRISSLRPQGLSNPSTLSAQPQLSRSHSSGSVLPLGELEGRRSTRDRR
SPAEPEGGPASEGAARPLPRFNSVPLTDTGHERQSEGSSSPQLQESVFHL
LVPSVILVLLAVGGLLFYRWRRRSHQEPQRADSPLEQPEGSPLTQDDRQV
ELPV.

Human M-CSF γ splice variant (SEQ ID NO: 2):

MTAPGAAGRCPPTTWLGSLLLLVCLLASRSITEEVSEYCSHMIGSGHLQS
LQRLIDSQMETSCQITFEFVDQEQLKDPVCYLKKAFLLVQDIMEDTMRFR
DNTPNAIAIVQLQELSLRLKSCFTKDYEEHDKACVRTFYETPLQLLEKVK
NVFNETKNLLDKDWNIFSKNCNNSFAECSSQDVVTKPDCNCLYPKAIPSS
DPASVSPHQPLAPSMAPVAGLTWEDSEGTEGSSLLPGEQPLHTVDPGSAK
QRPPRSTCQSFEPPETPVVKDSTIGGSPQPRPSVGAFNPGMEDILDSAMG
TNWVPEEASGEASEIPVPQGTELSPSRPGGGSMQTEPARPSNFLSASSPL
PASAKGQQPADVTGHERQSEGSSSPQLQESVFHLLVPSVILVLLAVGGLL
FYRWRRRSHQEPQRADSPLEQPEGSPLTQDDRQVELPV

Human M-CSF α splice variant (SEQ ID NO: 3):

MTAPGAAGRCPPTTWLGSLLLLVCLLASRSITEEVSEYCSHMIGSGHLQS
LQRLIDSQMETSCQITFEFVDQEQLKDPVCYLKKAFLLVQDIMEDTMRFR
DNTPNAIAIVQLQELSLRLKSCFTKDYEEHDKACVRTFYETPLQLLEKVK
NVFNETKNLLDKDWNIFSKNCNNSFAECSSQGHERQSEGSSSPQLQESVF
HLLVPSVILVLLAVGGLLFYRWRRRSHQEPQRADSPLEQPEGSPLTQDDR
QVELPV

Active fragments of M-CSF have been identified. See, e.g., Takahashi et al., Biochem. Biophys. Res. Comm 161(2): 892-901 (1989) and Yamanishi et al., J. Biochem 109: 404-409 (1991). In one embodiment, the present invention comprises providing to an animal in need thereof, an isolated M-CSF polypeptide fragment, where the polypeptide fragment comprises an amino acid sequence at least 90% similar to an M-CSF reference amino acid sequence, an amino acid sequence that is at least 95% similar to an M-CSF reference amino acid sequence or an amino acid sequence identical to an M-CSF reference amino acid sequence. According to this embodiment, M-CSF reference amino acid sequences include, but are not limited to:

(a) amino acid residues X to Y of SEQ ID NOS. 1, 2 or 3 wherein X is any integer from 33-37 and Y is any integer from 145-158;
(b) amino acid residues X to Y₁ of SEQ ID NOS. 1, 2 or 3, wherein Y₁ is any integer from 181-191;
(c) amino acid residues X to Y₂ of SEQ ID NOS. 1, 2 or 3, wherein Y₂ is any integer from 220-224;
(d) amino acid residues X to Y₃ of SEQ ID NOS. 1 or 2, wherein Y₃ is 337;
(e) amino acid residues X to Y₄ of SEQ ID NO. 1, wherein Y₄ is 554;
(f) amino acid residues X to Y₅ of SEQ ID NOS. 1 or 2, wherein Y₅ is 438;
(g) amino acid residues X to Y₆ of SEQ ID NOS. 1, 2 or 3, wherein Y₆ is 256;
(h) amino acid residues X₁ to Y of SEQ ID NOS. 1, 2 or 3, wherein X₁ is any integer from 1-4;
(i) amino acid residues X₁ to Y₁ of SEQ ID NOS. 1, 2 or 3, wherein Y₁ is any integer from 181-191;
(j) amino acid residues X₁ to Y₂ of SEQ ID NOS. 1, 2 or 3, wherein Y₂ is any integer from 220-224;
(k) amino acid residues X₁ to Y₃ of SEQ ID NOS. 1, 2 or 3, wherein Y₃ is 337;
(l) amino acid residues X₁ to Y₄ of SEQ ID NO. 1, wherein Y₄ is 554;
(m) amino acid residues X₁ to Y₅ of SEQ ID NOS. 1 or 2, wherein Y₅ is 438;
(n) amino acid residues X₁ to Y₆ of SEQ ID NOS. 1, 2 or 3, wherein Y₆ is 256.
In certain embodiments, M-CSF reference amino acid sequences include, but are not limited to:

### (i) 1-145 of SEQ ID NOS. 1, 2 or 3;

### (ii) 1-149 of SEQ ID NOS. 1, 2 or 3;

(iii) 1-150 of SEQ ID NOS. 1, 2 or 3;

### (iv) 1-158 of SEQ ID NOS. 1, 2 or 3;

### (v) 1-177 of SEQ ID NOS. 1, 2 or 3;

### (vi) 1-181 of SEQ ID NOS. 1, 2 or 3;

(vii) 1-182 of SEQ ID NOS. 1, 2 or 3;
(viii) 1-190 of SEQ ID NOS. 1, 2 or 3;

### (ix) 1-221 of SEQ ID NOS. 1, 2 or 3;

### (x) 1-223 of SEQ ID NOS. 1, 2 or 3;

### (xi) 1-377 of SEQ ID NOS. 1 or 2;

(xii) 33-177 of SEQ ID NOS. 1, 2 or 3;
(xiii) 33-181 of SEQ ID NOS. 1, 2 or 3;
(xiv) 33-182 of SEQ ID NOS. 1, 2 or 3;

### (xv) 33-190 of SEQ ID NOS. 1, 2 or 3;

(xvi) 33-221 of SEQ ID NOS. 1, 2 or 3;
(xvii) 33-223 of SEQ ID NOS. 1, 2 or 3;
(xviii) 33-377 of SEQ ID NOS. 1 or 2;
(ixx) 1-554 of SEQ ID NO. 1;

### (xx) 33-544 of SEQ ID NO. 1;

(xxi) 1-438 of SEQ ID NOS. 1 or 2;
(xxii) 33-438 of SEQ ID NOS. 1 or 2;
(xxiii) 1-256 of SEQ ID NOS. 1, 2 or 3;
(xxiv) 33-256 of SEQ ID NOS. 1, 2 or 3; and
(ixx) a combination of two or more of said reference amino acid sequences.

The M-CSF administered as part of the present invention may be a monomer or dimer. The dimer can be a homodimer composed of polypeptides having the above recited M-CSF reference amino acid sequences or a heterodimer composed of any two polypeptides having the above recited M-CSF reference amino acid sequences. The polypetides present in the homo- or heterodimer may be substituted M-CSF polypeptides or polypeptide fragments having at least 70%, 75%, 80%, 85%, 90%, or 95% identical to polypeptides of SEQ ID NOS:1, 2 or 3 or fragments thereof.

By “an M-CSF reference amino acid sequence,” or “reference amino acid sequence” is meant the specified sequence without the introduction of any amino acid substitutions. As one of ordinary skill in the art would understand, if there are no substitutions, the “isolated polypeptide” of the invention comprises an amino acid sequence which is identical to the reference amino acid sequence.

M-CSF polypeptides or polypeptide fragments that are, for example, at least 90% or at least 95% similar to the M-CSF reference amino acid sequence may contain amino acid substitutions. Such substitutions include substitution of, e.g., individual cysteine residues in the reference amino acid sequence with different amino acids. By substituting other amino acid residues for cysteine residues, disulfide linkages are blocked. Blocking disulfide linkages may affect the three dimensional shape of the polypeptide or interfere with the polypeptide's binding to a receptor molecule. Any different amino acid may be substituted for a cysteine in the reference amino acid sequence. Which different amino acid is used depends on a number of criteria, for example, the effect of the substitution on the conformation of the polypeptide fragment, the charge of the polypeptide fragment, or the hydrophilicity of the polypeptide fragment. Typical amino acids to substitute for cysteines in the reference amino acid include alanine, serine, threonine, in particular, alanine. Making such substitutions through engineering of a polynucleotide encoding the polypeptide fragment is well within the routine expertise of one of ordinary skill in the art.

In one aspect, this embodiment includes a polypeptide comprising two or more polypeptide fragments as described above in a fusion protein, as well as fusion proteins comprising a polypeptide fragment as described above fused to a heterologous amino acid sequence. The invention further encompasses variants, analogs, or derivatives of polypeptide fragments as described above.

Corresponding fragments of M-CSF polypeptides or polypeptide fragments at least 70%, 75%, 80%, 85%, 90%, or 95% identical to polypeptides of SEQ ID NOS:1, 2 or 3 described herein are also contemplated. As known in the art, “sequence identity” between two polypeptides is determined by comparing the amino acid sequence of one polypeptide to the sequence of a second polypeptide. When discussed herein, whether any particular polypeptide is at least about 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

Corresponding fragments of M-CSF polypeptides or polypeptide fragments at least 70%, 75%, 80%, 85%, 90%, or 95% similar to polypeptides of SEQ ID NOS:1, 2 or 3 described herein are also contemplated. As known in the art, “sequence similarity” between two polypeptides is determined by comparing the amino acid sequence of one polypeptide to the sequence of a second polypeptide.

In the present invention, a polypeptide can be composed of amino acids joined to each other by peptide bonds or modified peptide bonds, i.e., peptide isosteres, and may contain amino acids other than the 20 gene-encoded amino acids (e.g. non-naturally occurring amino acids). The polypeptides of the present invention may be modified by either natural, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T.E. Creighton, W.H. Freeman and Company, New York (1993); Posttranslational Covalent Modification of Proteins, B.C. Johnson, Ed., Academic Press, New York, pgs. 1-12 (1983); Seifter et al., Meth. Enzymol. 182:626-646 (1990); Rattan et al., Ann. NY Acad. Sci. 663:48-62 (1992)).

Polypeptides described herein may be cyclic. Cyclization of the polypeptides reduces the conformational freedom of linear peptides and results in a more structurally constrained molecule. Many methods of peptide cyclization are known in the art. For example, “backbone to backbone” cyclization by the formation of an amide bond between the N-terminal and the C-terminal amino acid residues of the peptide. The “backbone to backbone” cyclization method includes the formation of disulfide bridges between two ω-thio amino acid residues (e.g. cysteine, homocysteine). Certain peptides of the present invention include modifications on the N- and C-terminus of the peptide to form a cyclic polypeptide. Such modifications include, but are not limited, to cysteine residues, acetylated cysteine residues, cysteine residues with a NH₂ moiety and biotin. Other methods of peptide cyclization are described in Li & Roller. Curr. Top. Med. Chem. 3:325-341 (2002), which is incorporated by reference herein in its entirety.

In another embodiment, the present invention provides an isolated polypeptide with a first polypeptide fragment and a second polypeptide fragment, where the first polypeptide fragment comprises an amino acid sequence at least 90% similar to an M-CSF reference amino acid sequence and where the second polypeptide fragment comprises a fusion moiety.

In methods of the present invention, a growth factor, colony stimulating factor or M-CSF polypeptide or active variant, fragment or derivative thereof can be administered directly as a preformed polypeptide, or indirectly as described herein. In some embodiments of the invention, a growth factor, colony stimulating factor or M-CSF polypeptide or polypeptide fragment of the invention is administered in a treatment method that includes: (1) transforming or transfecting an implantable host cell with a nucleic acid, e.g., a vector that expresses a growth factor, colony stimulating factor or M-CSF polypeptide or polypeptide fragment of the invention; and (2) administering the transformed host cell to an animal at any site effective to allow for systemic administration. For example, the transformed host cell can be administered by orally, nasally, parenterally, transdermally, topically, intraocularly, intrabronchially, intraperitoneally, intravenously, subcutaneously, intramuscularly, buccally, sublingually, vaginally, intraheptically, intracardiac, intrapancreatic, transplantation, by inhalation or by an implanted pump.

In some embodiments of the invention, the host cell is removed from a animal, temporarily cultured, transformed or transfected with an isolated nucleic acid encoding a growth factor, colony stimulating factor or M-CSF polypeptide or polypeptide fragment of the invention, and implanted back into the same animal from which it was removed. The cell can be, but is not required to be, removed from the same site at which it is implanted. Such embodiments, sometimes known as ex vivo gene therapy, can provide a continuous supply of the growth factor, colony stimulating factor or M-CSF polypeptide or polypeptide fragment for a limited period of time.

Additional exemplary growth factors, colony stimulating factors or M-CSF polypeptides that can be administered in the method of the invention and methods and materials for obtaining these molecules for practicing the present invention are described below.

### Fusion Proteins and Conjugated Polypeptides

Some embodiments of the invention involve the use of a growth factor, colony stimulating factor or M-CSF polypeptide that is not the full-length growth factor, colony stimulating factor or M-CSF protein, e.g., polypeptide fragments of a growth factor, colony stimulating factor or M-CSF, fused to a heterologous polypeptide moiety to form a fusion protein. Such fusion proteins can be used to accomplish various objectives, e.g., increased serum half-life, improved bioavailability, in vivo targeting to a specific organ or tissue type, e.g., the brain, improved recombinant expression efficiency, improved host cell secretion, ease of purification, and higher avidity. Depending on the objective(s) to be achieved, the heterologous moiety can be inert or biologically active. Also, it can be chosen to be stably fused to the growth factor, colony stimulating factor or M-CSF polypeptide moiety or to be cleavable, in vitro or in vivo. Heterologous moieties to accomplish these other objectives are known in the art.

As an alternative to expression of a fusion protein, a chosen heterologous moiety can be preformed and chemically conjugated to the growth factor, colony stimulating factor or M-CSF polypeptide moiety. In most cases, a chosen heterologous moiety will function similarly, whether fused or conjugated to the growth factor, colony stimulating factor or M-CSF polypeptide moiety. Therefore, in the following discussion of heterologous amino acid sequences, unless otherwise noted, it is to be understood that the heterologous sequence can be joined to the growth factor, colony stimulating factor or M-CSF polypeptide moiety in the form of a fusion protein or as a chemical conjugate.

Growth factor, colony stimulating factor or M-CSF polypeptides for use in the treatment methods disclosed herein include derivatives that are modified, i.e., by the covalent attachment of any type of molecule such that covalent attachment does not prevent the growth factor, colony stimulating factor or M-CSF polypeptide from performing its required function. For example, but not by way of limitation, the growth factor, colony stimulating factor or M-CSF polypeptides of the present invention may be modified e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The heterologous polypeptide to which the growth factor, colony stimulating factor or M-CSF polypeptide is fused is useful therapeutically or is useful to target the growth factor, colony stimulating factor or M-CSF polypeptide. Growth factor, colony stimulating factor or M-CSF fusion proteins can be used to accomplish various objectives, e.g., increased serum half-life, improved bioavailability, in vivo targeting to a specific organ or tissue type, e.g., the brain, improved recombinant expression efficiency, improved host cell secretion, ease of purification, and higher avidity. Depending on the objective(s) to be achieved, the heterologous moiety can be inert or biologically active. Also, it can be chosen to be stably fused to the growth factor, colony stimulating factor or M-CSF polypeptide or to be cleavable, in vitro or in vivo. Heterologous moieties to accomplish these other objectives are known in the art.

Pharmacologically active polypeptides such as growth factor, colony stimulating factor or M-CSF polypeptides may exhibit rapid in vivo clearance, necessitating large doses to achieve therapeutically effective concentrations in the body. In addition, polypeptides smaller than about 60 kDa potentially undergo glomerular filtration, which sometimes leads to nephrotoxicity. Fusion or conjugation of relatively small polypeptides such as polypeptide fragments of the growth factor, colony stimulating factor or M-CSF signaling domain can be employed to reduce or avoid the risk of such nephrotoxicity. Various heterologous amino acid sequences, i.e., polypeptide moieties or “carriers,” for increasing the in vivo stability, i.e., serum half-life, of therapeutic polypeptides are known. Examples include serum albumins such as, e.g., bovine serum albumin (BSA) or human serum albumin (HSA).

Due to its long half-life, wide in vivo distribution, and lack of enzymatic or immunological function, essentially full-length human serum albumin,(HSA), or an HSA fragment, is commonly used as a heterologous moiety. Through application of methods and materials such as those taught in Yeh et al., Proc. Natl. Acad. Sci. USA, 89:1904-08 (1992) and Syed et al., Blood 89:3243-52 (1997), HSA can be used to form a fusion protein or polypeptide conjugate that displays pharmacological activity by virtue of the M-CSF polypeptide moiety while displaying significantly increased in vivo stability, e.g., 10-fold to 100-fold higher. The C-terminus of the HSA can be fused to the N-terminus of the growth factor, colony stimulating factor or M-CSF polypeptide moiety. Since HSA is a naturally secreted protein, the HSA signal sequence can be exploited to obtain secretion of the fusion protein into the cell culture medium when the fusion protein is produced in a eukaryotic, e.g., mammalian, expression system.

In certain embodiments, the growth factor, colony stimulating factor or M-CSF polypeptides for use in the methods of the present invention further comprise a targeting moiety. Targeting moieties include a protein or a peptide which directs localization to a certain part of the body.

Some embodiments of the invention employ a growth factor, colony stimulating factor or M-CSF polypeptide moiety fused to a hinge and Fc region, i.e., the C-terminal portion of an Ig heavy chain constant region. In some embodiments, amino acids in the hinge region may be substituted with different amino acids. Exemplary amino acid substitutions for the hinge region according to these embodiments include substitutions of individual cysteine residues in the hinge region with different amino acids. Any different amino acid may be substituted for a cysteine in the hinge region. Amino acid substitutions for the amino acids of the polypeptides of the invention and the reference amino acid sequence can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Typical amino acids to substitute for cysteines in the reference amino acid include alanine, serine, threonine, in particular, serine and alanine. Making such substitutions through engineering of a polynucleotide encoding the polypeptide fragment is well within the routine expertise of one of ordinary skill in the art.

Potential advantages of an M-CSF-polypeptide-Fc fusion (“immunofusin”) include solubility, in vivo stability, and multivalency, e.g., dimerization. The Fc region used can be an IgA, IgD, or IgG Fc region (hinge-CH2—CH3). Alternatively, it can be an IgE or IgM Fc region (hinge-CH2-CH3-CH4). An IgG Fc region is generally used, e.g., an IgG1 Fc region or IgG4 Fc region. Materials and methods for constructing and expressing DNA encoding Fe fusions are known in the art and can be applied to obtain fusions without undue experimentation. Some embodiments of the invention employ a fusion protein such as those described in Capon et al., U.S. Pat. Nos. 5,428,130 and 5,565,335. In certain embodiments of the invention a native M-CSF polypeptide, or fragment thereof occurs as a dimer with an M-CSF-polypeptide-Fc fusion.

The signal sequence is a polynucleotide that encodes an amino acid sequence that initiates transport of a protein across the membrane of the endoplasmic reticulum. Signal sequences useful for constructing an immunofusin include antibody light chain signal sequences, e.g., antibody 14.18 (Gillies et al., J. Immunol. Meth. 125:191-202 (1989)), antibody heavy chain signal sequences, e.g., the MOPC141 antibody heavy chain signal sequence (Sakano et al., Nature 286:5774 (1980)). Alternatively, other signal sequences can be used. See, e.g., Watson, Nucl. Acids Res. 12:5145 (1984). The signal peptide is usually cleaved in the lumen of the endoplasmic reticulum by signal peptidases. This results in the secretion of a immunofusin protein containing the Fc region and the growth factor, colony stimulating factor or M-CSF polypeptide moiety.

In some embodiments, the DNA sequence may encode a proteolytic cleavage site between the secretion cassette and the growth factor, colony stimulating factor or M-CSF polypeptide moiety. Such a cleavage site may provide, e.g., for the proteolytic cleavage of the encoded fusion protein, thus separating the Fc domain from the target protein. Useful proteolytic cleavage sites include amino acid sequences recognized by proteolytic enzymes such as trypsin, plasmin, thrombin, factor Xa, or enterokinase K.

The secretion cassette can be incorporated into a replicable expression vector. Useful vectors include linear nucleic acids, plasmids, phagemids, cosmids and the like. An exemplary expression vector is pdC, in which the transcription of the immunofusin DNA is placed under the control of the enhancer and promoter of the human cytomegalovirus. See, e.g., Lo et al., Biochim. Biophys. Acta 1088:712 (1991); and Lo et al., Protein Engineering 11:495-500 (1998). An appropriate host cell can be transformed or transfected with a DNA that encodes a M-CSF polypeptide or polypeptide fragment and used for the expression and secretion of the polypeptide. Host cells that are typically used include immortal hybridoma cells, myeloma cells, 293 cells, Chinese hamster ovary (CHO) cells, Hela cells, and COS cells.

Fully intact, wild-type Fc regions display effector functions that normally are unnecessary and undesired in an Fc fusion protein used in the methods of the present invention. Therefore, certain binding sites typically are deleted from the Fc region during the construction of the secretion cassette. For example, since coexpression with the light chain is unnecessary, the binding site for the heavy chain binding protein, Bip (Hendershot et al., Immunol. Today 8:111-14 (1987)), is deleted from the CH2 domain of the Fc region of IgE, such that this site does not interfere with the efficient secretion of the immunofusin. Transmembrane domain sequences, such as those present in IgM, also are generally deleted.

The IgG1 Fc region is most often used. Alternatively, the Fc region of the other subclasses of immunoglobulin gamma (gamma-2, gamma-3 and gamma-4) can be used in the secretion cassette. The IgG1 Fc region of immunoglobulin gamma-1 is generally used in the secretion cassette and includes at least part of the hinge region, the CH2 region, and the CH3 region. In some embodiments, the Fc region of immunoglobulin gamma-1 is a CH2-deleted-Fc, which includes part of the hinge region and the CH3 region, but not the CH2 region. A CH2-deleted-Fc has been described by Gillies et al., Hum. Antibod. Hybridomas 1:47 (1990). In some embodiments, the Fc region of one of IgA, IgD, IgE, or IgM, is used.

M-CSF-polypeptide-moiety-Fc fusion proteins can be constructed in several different configurations. In one configuration the C-terminus of the M-CSF polypeptide moiety is fused directly to the N-terminus of the Fc hinge moiety. In a slightly different configuration, a short polypeptide, e.g., 2-10 amino acids, is incorporated into the fusion between the N-terminus of the M-CSF polypeptide moiety and the C-terminus of the Fc moiety. Such a linker provides conformational flexibility, which may improve biological activity in some circumstances. If a sufficient portion of the hinge region is retained in the Fc moiety, the M-CSF-polypeptide-moiety-Fc fusion will dimerize, thus forming a divalent molecule. A homogeneous population of monomeric Fc fusions will yield monospecific, bivalent dimers. A mixture of two monomeric Fc fusions each having a different specificity will yield bispecific, bivalent dimers.

Any of a number of cross-linkers that contain a corresponding amino-reactive group and thiol-reactive group can be used to link a growth factor, colony stimulating factor or M-CSF polypeptide or polypeptide fragment of the invention to serum albumin. Examples of suitable linkers include amine reactive cross-linkers that insert a thiol-reactive maleimide, e.g., SMCC, AMAS, BMPS, MBS, EMCS, SMPB, SMPH, KMUS, and GMBS. Other suitable linkers insert a thiol-reactive haloacetate group, e.g., SBAP, SIA, SLAB. Linkers that provide a protected or non-protected thiol for reaction with sulfhydryl groups to product a reducible linkage include SPDP, SMPT, SATA, and SATP. Such reagents are commercially available (e.g., Pierce Chemical Company, Rockford, Ill.).

Conjugation does not have to involve the N-terminus of a growth factor, colony stimulating factor or M-CSF polypeptide or polypeptide fragment of the invention or the thiol moiety on serum albumin. For example, M-CSF-polypeptide-albumin fusions can be obtained using genetic engineering techniques, wherein the growth factor, colony stimulating factor or M-CSF polypeptide moiety is fused to the serum albumin gene at its N-terminus, C-terminus, or both.

Growth factors, colony stimulating factors or M-CSF polypeptides administered in the method of the invention can be fused to a polypeptide tag. The term “polypeptide tag,” as used herein, is intended to mean any sequence of amino acids that can be attached to, connected to, or linked to a growth factor, colony stimulating factor or M-CSF polypeptide and that can be used to identify, purify, concentrate or isolate the growth factor, colony stimulating factor or M-CSF polypeptide. The attachment of the polypeptide tag to the growth factor, colony stimulating factor or M-CSF polypeptide may occur, e.g., by constructing a nucleic acid molecule that comprises: (a) a nucleic acid sequence that encodes the polypeptide tag, and (b) a nucleic acid sequence that encodes a growth factor, colony stimulating factor or M-CSF polypeptide. Exemplary polypeptide tags include, e.g., amino acid sequences that are capable of being post-translationally modified, e.g., amino acid sequences that are biotinylated. Other Exemplary polypeptide tags include, e.g., amino acid sequences that are capable of being recognized and/or bound by an antibody (or fragment thereof) or other specific binding reagent. Polypeptide tags that are capable of being recognized by an antibody (or fragment thereof) or other specific binding reagent include, e.g., those that are known in the art as “epitope tags.” An epitope tag may be a natural or an artificial epitope tag. Natural and artificial epitope tags are known in the art, including, e.g., artificial epitopes such as FLAG, Strep, or poly-histidine peptides. FLAG peptides include the sequence Asp-Tyr-Lys-Asp-Asp-Asp-Asp-Lys (SEQ ID NO: 15) or Asp-Tyr-Lys-Asp-Glu-Asp-Asp-Lys (SEQ ID NO: 16) (Einhauer, A. and Jungbauer, A., J. Biochem. Biophys. Methods 49:1-3:455-465 (2001)). The Strep epitope has the sequence Ala-Trp-Arg-His-Pro-Gln-Phe-Gly-Gly (SEQ ID NO: 17). The VSV-G epitope can also be used and has the sequence Tyr-Thr-Asp-11e-Glu-Met-Asn-Arg-Leu-Gly-Lys (SEQ ID NO: 18). Another artificial epitope is a poly-His sequence having six histidine residues (His-His-His-His-His-His (SEQ ID NO: 19). Naturally-occurring epitopes include the influenza virus hemagglutinin (HA) sequence Tyr-Pro-Tyr-Asp-Val-Pro-Asp-Tyr-Ala-Ile-Glu-Gly-Arg (SEQ ID NO: 20) recognized by the monoclonal antibody 12CA5 (Murray et al., Anal. Biochem. 229:170-179 (1995)) and the eleven amino acid sequence from human c-myc (Myc) recognized by the monoclonal antibody 9E10 (Glu-Gln-Lys-Leu-Leu-Ser-Glu-Glu-Asp-Leu-Asn (SEQ ID NO: 21) (Manstein et al., Gene 162:129-134 (1995)). Another useful epitope is the tripeptide Glu-Glu-Phe which is recognized by the monoclonal antibody YL 1/2. (Stammers et al. FEBS Lett. 283:298-302 (1991)).

In certain embodiments, a growth factor, colony stimulating factor or M-CSF polypeptide and the polypeptide tag may be connected via a linking amino acid sequence. As used herein, a “linking amino acid sequence” may be an amino acid sequence that is capable of being recognized and/or cleaved by one or more proteases. Amino acid sequences that can be recognized and/or cleaved by one or more proteases are known in the art. Exemplary amino acid sequences are those that are recognized by the following proteases: factor VIIa, factor IXa, factor Xa, APC, t-PA, u-PA, trypsin, chymotrypsin, enterokinase, pepsin, cathepsin B,H,L,S,D, cathepsin G, renin, angiotensin converting enzyme, matrix metalloproteases (collagenases, stromelysins, gelatinases), macrophage elastase, Cir, and C is. The amino acid sequences that are recognized by the aforementioned proteases are known in the art. Exemplary sequences recognized by certain proteases can be found, e.g., in U.S. Pat. No. 5,811,252.

Polypeptide tags can facilitate purification using commercially available chromatography media.

In some embodiments of the invention, a growth factor, colony stimulating factor or M-CSF polypeptide fusion construct is used to enhance the production of a growth factor, colony stimulating factor or M-CSF polypeptide moiety in bacteria. In such constructs, a bacterial protein normally expressed and/or secreted at a high level is employed as the N-terminal fusion partner of a growth factor, colony stimulating factor or M-CSF polypeptide or polypeptide fragment of the invention. See, e.g., Smith et al., Gene 67:31 (1988); Hopp et al., Biotechnology 6:1204 (1988); La Vallie et al., Biotechnology 11:187 (1993).

By fusing a growth factor, colony stimulating factor or M-CSF polypeptide moiety at the amino and carboxy termini of a suitable fusion partner, bivalent or tetravalent forms of a growth factor, colony stimulating factor or M-CSF polypeptide or polypeptide fragment can be obtained for administration according to the method of the invention. For example, a growth factor, colony stimulating factor or M-CSF polypeptide moiety can be fused to the amino and carboxy termini of an Ig moiety to produce a bivalent monomeric polypeptide containing two growth factor, colony stimulating factor or M-CSF polypeptide moieties. Upon dimerization of two of these monomers, by virtue of the Ig moiety, a tetravalent form of a growth factor, colony stimulating factor or M-CSF polypeptide is obtained. Such multivalent forms can be used to achieve increased binding affinity for the target. Multivalent forms of a growth factor, colony stimulating factor or M-CSF polypeptide or polypeptide fragment of the invention also can be obtained by placing growth factor, colony stimulating factor or M-CSF polypeptide moieties in tandem to form concatamers, which can be employed alone or fused to a fusion partner such as Ig or HSA.

Conjugated Polymers (Other than Polypeptides)

Some embodiments of the invention involve a growth factor polypeptide or polypeptide fragment wherein one or more polymers are conjugated (covalently linked) to the growth factor polypeptide. Some embodiments of the invention involve a growth factor, colony stimulating factor or M-CSF polypeptide or polypeptide fragment wherein one or more polymers are conjugated (covalently linked) to the growth factor, colony stimulating factor or M-CSF polypeptide. Examples of polymers suitable for such conjugation include polypeptides (discussed above), sugar polymers and polyalkylene glycol chains. Typically, but not necessarily, a polymer is conjugated to the growth factor, colony stimulating factor or M-CSF polypeptide or polypeptide fragment of the invention for the purpose of improving one or more of the following: solubility, stability, or bioavailability.

The class of polymer generally used for conjugation to a growth factor, colony stimulating factor or M-CSF polypeptide or polypeptide fragment of the invention is a polyalkylene glycol. Polyethylene glycol (PEG) is most frequently used. PEG moieties, e.g., 1, 2, 3, 4 or 5 PEG polymers, can be conjugated to each growth factor, colony stimulating factor or M-CSF polypeptide to increase serum half life, as compared to the growth factor, colony stimulating factor or M-CSF polypeptide alone. PEG moieties are non-antigenic and essentially biologically inert. PEG moieties used in the practice of the invention may be branched or unbranched.

The number of PEG moieties attached to the growth factor, colony stimulating factor or M-CSF polypeptide and the molecular weight of the individual PEG chains can vary. In general, the higher the molecular weight of the polymer, the fewer polymer chains attached to the polypeptide. Usually, the total polymer mass attached to a growth factor, colony stimulating factor or M-CSF polypeptide or polypeptide fragment is from 20 kDa to 40 kDa. Thus, if one polymer chain is attached, the molecular weight of the chain is generally 20-40 kDa. If two chains are attached, the molecular weight of each chain is generally 10-20 kDa. If three chains are attached, the molecular weight is generally 7-14 kDa.

The polymer, e.g., PEG, can be linked to the growth factor, colony stimulating factor or M-CSF polypeptide through any suitable, exposed reactive group on the polypeptide. The exposed reactive group(s) can be, e.g., an N-terminal amino group or the epsilon amino group of an internal lysine residue, or both. An activated polymer can react and covalently link at any free amino group on the growth factor, colony stimulating factor or M-CSF polypeptide. Free carboxylic groups, suitably activated carbonyl groups, hydroxyl, guanidyl, imidazole, oxidized carbohydrate moieties and mercapto groups of the v polypeptide (if available) also can be used as reactive groups for polymer attachment.

In a conjugation reaction, from about 1.0 to about 10 moles of activated polymer per mole of polypeptide, depending on polypeptide concentration, is typically employed. Usually, the ratio chosen represents a balance between maximizing the reaction while minimizing side reactions (often non-specific) that can impair the desired pharmacological activity of the growth factor, colony stimulating factor or M-CSF polypeptide moiety. Preferably, at least 50% of the biological activity (as demonstrated, e.g., in any of the assays described herein or known in the art) of the growth factor, colony stimulating factor or M-CSF polypeptide is retained, and most preferably nearly 100% is retained.

The polymer can be conjugated to the growth factor, colony stimulating factor or M-CSF polypeptide using conventional chemistry. For example, a polyalkylene glycol moiety can be coupled to a lysine epsilon amino group of the growth factor, colony stimulating factor or M-CSF polypeptide.

Linkage to the lysine side chain can be performed with an N-hydroxylsuccinimide (NHS) active ester such as PEG succinimidyl succinate (SS-PEG) and succinimidyl propionate (SPA-PEG). Suitable polyalkylene glycol moieties include, e.g., carboxymethyl-NHS and norleucine-NHS, SC. These reagents are commercially available. Additional amine-reactive PEG linkers can be substituted for the succinimidyl moiety. These include, e.g., isothiocyanates, nitrophenylcarbonates (PNP), epoxides, benzotriazole carbonates, SC-PEG, tresylate, aldehyde, epoxide, carbonylimidazole and PNP carbonate. Conditions are usually optimized to maximize the selectivity and extent of reaction. Such optimization of reaction conditions is within ordinary skill in the art.

PEGylation can be carried out by any of the PEGylation reactions known in the art. See, e.g., Focus on Growth Factors, 3: 4-10, 1992 and European patent applications EP 0 154 316 and EP 0 401 384. PEGylation may be carried out using an acylation reaction or an alkylation reaction with a reactive polyethylene glycol molecule (or an analogous reactive water-soluble polymer).

PEGylation by acylation generally involves reacting an active ester derivative of polyethylene glycol. Any reactive PEG molecule can be employed in the PEGylation. PEG esterified to N-hydroxysuccinimide (NHS) is a frequently used activated PEG ester. As used herein, “acylation” includes without limitation the following types of linkages between the therapeutic protein and a water-soluble polymer such as PEG: amide, carbamate, urethane, and the like. See, e.g., Bioconjugate Chem. 5: 133-140, 1994. Reaction parameters are generally selected to avoid temperature, solvent, and pH conditions that would damage or inactivate the growth factor, colony stimulating factor or M-CSF polypeptide.

Generally, the connecting linkage is an amide and typically at least 95% of the resulting product is mono-, di- or tri-PEGylated. However, some species with higher degrees of PEGylation may be formed in amounts depending on the specific reaction conditions used. Optionally, purified PEGylated species are separated from the mixture, particularly unreacted species, by conventional purification methods, including, e.g., dialysis, salting-out, ultrafiltration, ion-exchange chromatography, gel filtration chromatography, hydrophobic exchange chromatography, and electrophoresis.

PEGylation by alkylation generally involves reacting a terminal aldehyde derivative of PEG with a growth factor, colony stimulating factor or M-CSF polypeptide or polypeptide fragment of the invention in the presence of a reducing agent. In addition, one can manipulate the reaction conditions to favor PEGylation substantially only at the N-terminal amino group of the M-CSF polypeptide, i.e. a mono-PEGylated protein. In either case of mono-PEGylation or poly-PEGylation, the PEG groups are typically attached to the protein via a —CH2-NH— group. With particular reference to the —CH2- group, this type of linkage is known as an “alkyl” linkage.

Derivatization via reductive alkylation to produce an N-terminally targeted mono-PEGylated product exploits differential reactivity of different types of primary amino groups (lysine versus the N-terminal) available for derivatization. The reaction is performed at a pH that allows one to take advantage of the pKa differences between the epsilon-amino groups of the lysine residues and that of the N-terminal amino group of the protein. By such selective derivatization, attachment of a water-soluble polymer that contains a reactive group, such as an aldehyde, to a protein is controlled: the conjugation with the polymer takes place predominantly at the N-terminus of the protein and no significant modification of other reactive groups, such as the lysine side chain amino groups, occurs.

The polymer molecules used in both the acylation and alkylation approaches are selected from among water-soluble polymers. The polymer selected is typically modified to have a single reactive group, such as an active ester for acylation or an aldehyde for alkylation, so that the degree of polymerization may be controlled as provided for in the present methods. An exemplary reactive PEG aldehyde is polyethylene glycol propionaldehyde, which is water stable, or mono C1-C10 alkoxy or aryloxy derivatives thereof (see, e.g., Harris et al., U.S. Pat. No. 5,252,714). The polymer may be branched or unbranched. For the acylation reactions, the polymer(s) selected typically have a single reactive ester group. For reductive alkylation, the polymer(s) selected typically have a single reactive aldehyde group. Generally, the water-soluble polymer will not be selected from naturally occurring glycosyl residues, because these are usually made more conveniently by mammalian recombinant expression systems.

Methods for preparing a PEGylated growth factor, colony stimulating factor or M-CSF polypeptides of the invention generally includes the steps of (a) reacting a growth factor, colony stimulating factor or M-CSF polypeptide or polypeptide fragment of the invention with polyethylene glycol (such as a reactive ester or aldehyde derivative of PEG) under conditions whereby the molecule becomes attached to one or more PEG groups, and (b) obtaining the reaction product(s). In general, the optimal reaction conditions for the acylation reactions will be determined case-by-case based on known parameters and the desired result. For example, a larger the ratio of PEG to protein, generally leads to a greater the percentage of poly-PEGylated product.

Reductive alkylation to produce a substantially homogeneous population of mono-polymer/growth factor, colony stimulating factor or M-CSF polypeptide generally includes the steps of: (a) reacting a growth factor, colony stimulating factor or M-CSF polypeptide or polypeptide fragment of the invention with a reactive PEG molecule under reductive alkylation conditions, at a pH suitable to permit selective modification of the N-terminal amino group of the growth factor, colony stimulating factor or M-CSF; and (b) obtaining the reaction product(s).

For a substantially homogeneous population of mono-polymer/growth factor, colony stimulating factor or M-CSF polypeptide, the reductive alkylation reaction conditions are those that permit the selective attachment of the water-soluble polymer moiety to the N-terminus of a growth factor, colony stimulating factor or M-CSF polypeptide or polypeptide fragment of the invention. Such reaction conditions generally provide for pKa differences between the lysine side chain amino groups and the N-terminal amino group. For purposes of the present invention, the pH is generally in the range of 3-9, typically 3-6.

Growth factors, colony stimulating factors, or M-CSF polypeptides of the invention can include a tag, e.g., a moiety that can be subsequently released by proteolysis. Thus, the lysine moiety can be selectively modified by first reacting a His-tag modified with a low-molecular-weight linker such as Traut's reagent (Pierce Chemical Company, Rockford, Ill.) which will react with both the lysine and N-terminus, and then releasing the His tag. The polypeptide will then contain a free SH group that can be selectively modified with a PEG containing a thiol-reactive head group such as a maleimide group, a vinylsulfone group, a haloacetate group, or a free or protected SH.

Traut's reagent can be replaced with any linker that will set up a specific site for PEG attachment. For example, Traut's reagent can be replaced with SPDP, SMPT, SATA, or SATP (Pierce Chemical Company, Rockford, Ill.). Similarly one could react the protein with an amine-reactive linker that inserts a maleimide (for example SMCC, AMAS, BMPS, MBS, EMCS, SMPB, SMPH, KMUS, or GMBS), a haloacetate group (SBAP, SIA, SIAB), or a vinylsulfone group and react the resulting product with a PEG that contains a free SH.

In some embodiments, the polyalkylene glycol moiety is coupled to a cysteine group of the growth factor, colony stimulating factor or M-CSF polypeptide. Coupling can be effected using, e.g., a maleimide group, a vinylsulfone group, a haloacetate group, or a thiol group.

Optionally, the growth factor, colony stimulating factor or M-CSF polypeptide is conjugated to the polyethylene-glycol moiety through a labile bond. The labile bond can be cleaved in, e.g., biochemical hydrolysis, proteolysis, or sulfhydryl cleavage. For example, the bond can be cleaved under in vivo (physiological) conditions.

The reactions may take place by any suitable method used for reacting biologically active materials with inert polymers, generally at about pH 5-8, e.g., pH 5, 6, 7, or 8, if the reactive groups are on the alpha amino group at the N-terminus. Generally the process involves preparing an activated polymer and thereafter reacting the protein with the activated polymer to produce the soluble protein suitable for formulation.

### Polynucleotides

The present invention also includes the administration of isolated polynucleotides that encode any one of the growth factor, colony stimulating factor or M-CSF polypeptides described herein. This includes polynucleotides that hybridize under moderately stringent or high stringency conditions to the noncoding strand, or complement, of a polynucleotide that encodes any one of the growth factor, colony stimulating factor or M-CSF polypeptides described herein. Stringent conditions are known to those skilled in the art and can be found in CURRENT PROTOCOLS IN MOLECULAR BIOLOGY, John Wiley & Sons, N.Y. (1989), 6.3.1-6.3.6.

The human M-CSF long (beta) intermediate (gamma) and short (alpha) polynucleotides are shown below as SEQ ID NOS. 4, 5 and 6, respectively.

Full-Length Human M-CSF polynucleotide (SEQ ID NO: 4):

aaagatccag tgtgctacct taagaaggca tttctcctgg
tacaatacat aatggaggac accatgcgct tcagagataa
cacccccaat gccatcgcca ttgtgcagct gcaggaactc
tctttgaggc tgaagagctg cttcaccaag gattatgaag
agcatgacaa ggcctgcgtc cgaactttct atgagacacc
tctccagttg ctggagaagg tcaagaatgt ctttaatgaa
acaaagaatc tccttgacaa ggactggaat attttcagca
agaactgcaa caacagcttt gctgaatgct ccagccaagg
ccatgagagg cagtccgagg gatcctccag cccgcagctc
caggagtctg tcttccacct gctggtgccc agtgtcatcc
tggtcttgct ggccgtcgga ggcctcttgt tctacaggtg
gaggcggcgg agccatcaag agcctcagag agcggattct
cccttggagc aaccagaggg cagccccctg actcaggatg
acagacaggt ggaactgcca gtgtagaggg aattctaaga
cccctcacca tcctggacac actcgtttgt caatgtccct
ctgaaaatgt gacgcccagc cccggacaca gtactccaga
tgttgtctga ccagctcaga gagagtacag tgggactgtt
accttccttg atatggacag tattcttcta tttgtgcaga
ttaagattgc attagttttt ttcttaacaa ctgcatcata
ctgttgtcat atgttgagcc tgtggtctat taaaacccct
agttccattt cccataaact tctgtcaagc cagaccatct
ctaccctgta cttggacaac ttaacttttt taaccaaagt
gcagtttatg ttcacctttg ttaaagccac cttgtggttt
ctgcccatca cctgaaccta ctgaagttgt gtgaaatcct
aattctgtca tctccgtagc cctcccagtt gtgcctcctg
cacattgatg agtgcctgct gttgtctttg cccatgttgt
tgatgtagct gtgaccctat tgttcctcac ccctgccccc
cgccaacccc agctggccca cctcttcccc ctcccaccca
agcccacagc cagcccatca ggaagccttc ctggcttctc
cacaaccttc tgactgctct tttcagtcat gcccctcctg
ctcttttgta tttggctaat agtatatcaa tttgcactt

Human M-CSF γ polynucleotide (SEQ ID NO: 5):

gagggctggc cagtgaggct cggcccgggg aaagtgaaag
tttgcctggg tcctctcggc gccagagccg ctctccgcat
cccaggacag cggtgcggcc ctcggccggg gcgcccactc
cgcagcagcc agcgagcgag cgagcgagcg agggcggccg
acgcgcccgg ccgggaccca gctgcccgta tgaccgcgcc
gggcgccgcc gggcgctgcc ctcccacgac atggctgggc
tccctgctgt tgttggtctg tctcctggcg agcaggagta
tcaccgagga ggtgtcggag tactgtagcc acatgattgg
gagtggacac ctgcagtctc tgcagcggct gattgacagt
cagatggaga cctcgtgcca aattacattt gagtttgtag
accaggaaca gttgaaagat ccagtgtgct accttaagaa
ggcatttctc ctggtacaag acataatgga ggacaccatg
cgcttcagag ataacacccc caatgccatc gccattgtgc
agctgcagga actctctttg aggctgaaga gctgcttcac
caaggattat gaagagcatg acaaggcctg cgtccgaact
ttctatgaga cacctctcca gttgctggag aaggtcaaga
atgtctttaa tgaaacaaag aatctccttg acaaggactg
gaatattttc agcaagaact gcaacaacag ctttgctgaa
tgctccagcc aagatgtggt gaccaagcct gattgcaact
gcctgtaccc caaagccatc cctagcagtg acccggcctc
tgtctcccct catcagcccc tcgccccctc catggcccct
gtggctggct tgacctggga ggactctgag ggaactgagg
gcagctccct cttgcctggt gagcagcccc tgcacacagt
ggatccaggc agtgccaagc agcggccacc caggagcacc
tgccagagct ttgagccgcc agagacccca gttgtcaagg
acagcaccat cggtggctca ccacagcctc gcccctctgt
cggggccttc aaccccggga tggaggatat tcttgactct
gcaatgggca ctaattgggt cccagaagaa gcctctggag
aggccagtga gattcccgta ccccaaggga cagagctttc
cccctccagg ccaggagggg gcagcatgca gacagagccc
gccagaccca gcaacttcct ctcagcatct tctccactcc
ctgcatcagc aaagggccaa cagccggcag atgtaactgg
ccatgagagg cagtccgagg gatcctccag cccgcagctc
caggagtctg tcttccacct gctggtgccc agtgtcatcc
tggtcttgct ggccgtcgga ggcctcttgt tctacaggtg
gaggcggcgg agccatcaag agcctcagag agcggattct
cccttggagc aaccagaggg cagccccctg actcaggatg
acagacaggt ggaactgcca gtgtagaggg aattctaag

Human M-CSF α polynucleotide (SEQ ID NO: 6):

gagggctggc cagtgaggct cggcccgggg aaagtgaaag
tttgcctggg tcctctcggc gccagagccg ctctccgcat
cccaggacag cggtgcggcc ctcggccggg gcgcccactc
cgcagcagcc agcgagcgag cgagcgagcg agggcggccg
acgcgcccgg ccgggaccca gctgcccgta tgaccgcgcc
gggcgccgcc gggcgctgcc ctcccacgac atggctgggc
tccctgctgt tgttggtctg tctcctggcg agcaggagta
tcaccgagga ggtgtcggag tactgtagcc acatgattgg
gagtggacac ctgcagtctc tgcagcggct gattgacagt
cagatggaga cctcgtgcca aattacattt gagtttgtag
accaggaaca gttgaaagat ccagtgtgct accttaagaa
ggcatttctc ctggtacaag acataatgga ggacaccatg
cgcttcagag ataacacccc caatgccatc gccattgtgc
agctgcagga actctctttg aggctgaaga gctgcttcac
caaggattat gaagagcatg acaaggcctg cgtccgaact
ttctatgaga cacctctcca gttgctggag aaggtcaaga
atgtctttaa tgaaacaaag aatctccttg acaaggactg
gaatattttc agcaagaact gcaacaacag ctttgctgaa
tgctccagcc aaggccatga gaggcagtcc gagggatcct
ccagcccgca gctccaggag tctgtcttcc acctgctggt
gcccagtgtc atcctggtct tgctggccgt cggaggcctc
ttgttctaca ggtggaggcg gcggagccat caagagcctc
agagagcgga ttctcccttg gagcaaccag agggcagccc
cctgactcag gatgacagac aggtggaact gccagtgtag
agggaattct aagctggacg cacagaacag tctctccgtg
ggaggagaca ttatggggcg tccaccacca cccctccctg
gccatcctcc tggaatgtgg tctgccctcc accagagctc
ctgcctgcca ggactggacc agagcagcca ggctggggcc
cctctgtctc aacccgcaga cccttgactg aatgagagag
gccagaggat gctccccatg ctgccactat ttattgtgag
ccctggaggc tcccatgtgc ttgaggaagg ctggtgagcc
cggctcagga ccctcttccc tcaggggctg caccctcctc
tcactccctt ccatgccgga acccaggcca gggacccacc
ggcctgtggt ttgtgggaaa gcagggtgga cgctgaggag
tgaaagaacc ctgcacccag agggcctgcc tggtgccaag
gtatcccagc ctggacaggc atggacctgt ctccagagag
aggagcctga agttcgtggg gcgggacagc gtcggcctga
tttcccgtaa aggtgtgcag cctgagagac gggaagagga
ggcctctgga cctgctggtc tgcactgaca gcctgaaggg
tctacaccct cggctcacct aagtgccctg tgctggttgc
caggcgcaga ggggaggcca gccctgccct caggacctgc
ctgacctgcc agtgatgcca agagggggat caagcactgg
cctctgcccc tcctccttcc agcacctgcc agagcttctc
caggaggcca agcagaggct cccctcatga aggaagccat
tgcactgtga acactgtacc tgcctgctga acagcctgcc
cccgtccatc catgagccag catccgtccg tcctccactc
tccagcctct cccca

The growth factor, colony stimulating factor or M-CSF encoding nucleic acids may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can employ any of the art known labels to obtain a labeled encoding nucleic acid molecule.

### Vectors of the Invention

Vectors comprising nucleic acids encoding the growth factor, colony stimulating factor or M-CSF polypeptides may be used to produce soluble polypeptides for use in the methods of the invention. The choice of vector and expression control sequences to which such nucleic acids are operably linked depends on the functional properties desired, e.g., protein expression, and the host cell to be transformed.

In a typical embodiment, a growth factor, colony stimulating factor or M-CSF polypeptide useful in the methods described herein is a recombinant protein produced by a cell (e.g., a CHO cell) that carries an exogenous nucleic acid encoding the protein. In other embodiments, the recombinant polypeptide is produced by a process commonly known as gene activation, wherein a cell that carries an exogenous nucleic acid that includes a promoter or enhancer is operably linked to an endogenous nucleic acid that encodes the polypeptide.

Routine techniques for making recombinant polypeptides (e.g., growth factor, colony stimulating factor or M-CSF polypeptides) may be used to construct expression vectors encoding the polypeptides of interest using appropriate transcriptional/translational control signals and the protein coding sequences. (See, for example, Sambrook et al., Molecular Cloning: A Laboratory Manual, 3d Ed. (Cold Spring Harbor Laboratory 2001)). These methods may include in vitro recombinant DNA and synthetic techniques and in vivo recombination, e.g., in vivo homologous recombination. Expression of a nucleic acid sequence encoding a polypeptide may be regulated by a second nucleic acid sequence that is operably linked to the polypeptide encoding sequence such that the polypeptide is expressed in a host transformed with the recombinant DNA molecule.

Expression control elements useful for regulating the expression of an operably linked coding sequence are known in the art. Examples include, but are not limited to, inducible promoters, constitutive promoters, secretion signals, and other regulatory elements. When an inducible promoter is used, it can be controlled, e.g., by a change in nutrient status, or a change in temperature, in the host cell medium.

Expression vectors capable of being replicated in a bacterial or eukaryotic host comprising a nucleic acid encoding a polypeptide are used to transfect a host and thereby direct expression of such nucleic acid to produce the polypeptide, which may then be isolated. The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Routine techniques for transfecting cells with exogenous DNA sequences may be used in the present invention. Transfection methods may include chemical means, e.g., calcium phosphate, DEAE-dextran, or liposome; or physical means, e.g., microinjection or electroporation. The transfected cells are grown up by routine techniques. For examples, see Kuchler et al. (1977) Biochemical Methods in Cell Culture and Virology. The expression products are isolated from the cell medium in those systems where the protein is secreted from the host cell, or from the cell suspension after disruption of the host cell system by, e.g., routine mechanical, chemical, or enzymatic means.

These methods may also be carried out using cells that have been genetically modified by other procedures, including gene targeting and gene activation (see Treco et al. WO 95/31560, herein incorporated by reference; see also Selden et al. WO 93/09222).

The vector can include a prokaryotic replicon, i.e., a DNA sequence having the ability to direct autonomous replication and maintenance of the recombinant DNA molecule extra-chromosomally in a bacterial host cell. Such replicons are well known in the art. In addition, vectors that include a prokaryotic replicon may also include a gene whose expression confers a detectable marker such as a drug resistance. Examples of bacterial drug-resistance genes are those that confer resistance to ampicillin or tetracycline.

Vectors that include a prokaryotic replicon can also include a prokaryotic or bacteriophage promoter for directing expression of the coding gene sequences in a bacterial host cell. Promoter sequences compatible with bacterial hosts are typically provided in plasmid vectors containing convenient restriction sites for insertion of a DNA segment to be expressed. Examples of such plasmid vectors are pUC8, pUC9, pBR322 and pBR329 (BioRad), pPL and pKK223 (Pharmacia). Any suitable prokaryotic host can be used to express a recombinant DNA molecule encoding a protein used in the methods of the invention.

For the purposes of this invention, numerous expression vector systems may be employed. For example, one class of vector utilizes DNA elements which are derived from animal viruses such as bovine papilloma virus, polyoma virus, adenovirus, adeno-associated virus, herpes simplex virus-1, vaccinia virus, baculovirus, retroviruses (RSV, MMTV or MOMLV) or SV40 virus. Examples of such vectors can be found in PCT publications WO 2006/060089 and WO2002/056918 which are incorporated herein in their entireties. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. The neomycin phosphotransferase (neo) gene is an example of a selectable marker gene (Southern et al., J. Mol. Anal. Genet. 1:327-341 (1982)). Additional elements may also be needed for optimal synthesis of mRNA. These elements may include signal sequences, splice signals, as well as transcriptional promoters, enhancers, and termination signals.

In one embodiment, a proprietary expression vector of Biogen IDEC, Inc., referred to as NEOSPLA (U.S. Pat. No. 6,159,730) may be used. This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression upon transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). Additional eukaryotic cell expression vectors are known in the art and are commercially available. Typically, such vectors contain convenient restriction sites for insertion of the desired DNA segment. Exemplary vectors include pSVL and pKSV-10 (Pharmacia), pBPV-1, pml2d (International Biotechnologies), pTDT1 (ATCC 31255), retroviral expression vector pMIG and pLL3.7, adenovirus shuttle vector pDC315, and AAV vectors. Other exemplary vector systems are disclosed e.g., in U.S. Pat. No. 6,413,777.

In general, screening large numbers of transformed cells for those which express suitably high levels of the antagonist is routine experimentation which can be carried out, for example, by robotic systems.

The recombinant expression vectors may carry sequences that regulate replication of the vector in host cells (e.g., origins of replication) and selectable marker genes. It will be appreciated by those skilled in the art that the design of the expression vector, including the selection of regulatory sequences may depend on such factors as the choice of the host cell to be transformed, the level of expression of protein desired, etc. Frequently used regulatory sequences for mammalian host cell expression include viral elements that direct high levels of protein expression in mammalian cells, such as promoters and enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (Adm1P)), polyoma and strong mammalian promoters such as native immunoglobulin and actin promoters. For further description of viral regulatory elements, and sequences thereof, see e.g., Stinski, U.S. Pat. No. 5,168,062; Bell, U.S. Pat. No. 4,510,245; and Schaffner, U.S. Pat. No. 4,968,615.

The selectable marker gene facilitates selection of host cells into which the vector has been introduced (see, e.g., Axel, U.S. Pat. Nos. 4,399,216; 4,634,665 and 5,179,017). For example, typically the selectable marker gene confers resistance to a drug, such as G418, hygromycin or methotrexate, on a host cell into which the vector has been introduced. Frequently used selectable marker genes include the dihydrofolate reductase (DHFR) gene (for use in dhfr-host cells with methotrexate selection/amplification) and the neo gene (for G418 selection).

Vectors comprising polynucleotides encoding growth factor, colony stimulating factor or M-CSF polypeptides can be used for transformation of a suitable host cell. Transformation can be by any suitable method. Methods for introduction of exogenous DNA into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

Transformation of host cells can be accomplished by conventional methods suited to the vector and host cell employed. For transformation of prokaryotic host cells, electroporation and salt treatment methods can be employed (Cohen et al., Proc. Natl. Acad. Sci. USA 69:2110-14 (1972)). For transformation of vertebrate cells, electroporation, cationic lipid or salt treatment methods can be employed. See, e.g., Graham et al., Virology 52:456-467 (1973); Wigler et al., Proc. Natl. Acad. Sci. USA 76:1373-76 (1979).

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to NSO, SP2 cells, baby hamster kidney (BHK) cells, monkey kidney cells (COS), human hepatocellular carcinoma cells (e.g., Hep G2), A549 cells DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3×63-Ag3.653 (mouse myeloma), BFA-1c1BPT (bovine endothelial cells), RAJI (human lymphocyte) and 293 (human kidney). Host cell lines are typically available from commercial services, the American Tissue Culture Collection or from published literature.

Expression of polypeptides from production cell lines can be enhanced using known techniques. For example, the glutamine synthetase (GS) system is commonly used for enhancing expression under certain conditions. See, e.g., European Patent Nos. 0 216 846, 0 256 055, and 0 323 997 and European Patent Application No. 89303964.4.

In some embodiments, the invention provides recombinant DNA molecules (rDNA) that contain a coding sequence. As used herein, a rDNA molecule is a DNA molecule that has been subjected to molecular manipulation. Methods for generating rDNA molecules are well known in the art, for example, see Sambrook et al., Molecular Cloning—A Laboratory Manual, Cold Spring Harbor Laboratory Press (1989). In some rDNA molecules, a coding DNA sequence is operably linked to expression control sequences and vector sequences. A vector of the present invention may be at least capable of directing the replication or insertion into the host chromosome, and preferably also expression, of the structural gene included in the rDNA molecule.

Expression vectors compatible with eukaryotic cells, preferably those compatible with vertebrate cells, can also be used to form a rDNA molecules that contains a coding sequence. Eukaryotic cell expression vectors are well known in the art and are available from several commercial sources. Typically, such vectors are provided containing convenient restriction sites for insertion of the desired DNA segment. Examples of such vectors are pSVL and pKSV-10 (Pharmacia), pBPV-1, pML2d (International Biotechnologies), pTDT1 (ATCC® 31255) and other eukaryotic expression vectors.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. (Southern et al., J. Mol. Anal. Genet. 1:327-341 (1982)). Alternatively, the selectable marker can be present on a separate plasmid, the two vectors introduced by co-transfection of the host cell, and transfectants selected by culturing in the appropriate drug for the selectable marker.

Other embodiments of the invention use a lentiviral vector for expression of the polynucleotides of the invention. Lentiviruses can infect noncycling and postmitotic cells, and also provide the advantage of not being silenced during development allowing generation of transgenic animals through infection of embryonic stem cells. Milhavet et al., Pharmacological Rev. 55:629-648 (2003). Other polynucleotide expressing viral vectors can be constructed based on, but not limited to, adeno-associated virus, retrovirus, adenovirus, or alphavirus.

Transcription of the polynucleotides of the invention can be driven from a promoter for eukaryotic RNA polymerase I (pol I), RNA polymerase II (pol II), or RNA polymerase III (pol III). Transcripts from pol H or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, Proc. Natl. Acad. Sci. USA 87:6743-7 (1990); Gao and Huang, Nucleic Acids Res. 21:2867-72 (1993); Lieber et al., Methods Enzymol. 217:47-66 (1993); Zhou et al., Mol. Cell. Biol. 10:4529-37 (1990)). Several investigators have demonstrated that polynucleotides expressed from such promoters can function in mammalian cells (e.g. Kashani-Sabet et al., Antisense Res. Dev. 2:3-15 (1992); Ojwang et al., Proc. Natl. Acad. Sci. USA 89:10802-6 (1992); Chen et al., Nucleic Acids Res. 20:4581-9 (1992); Yu et al., Proc. Natl. Acad. Sci. USA 90:6340-4 (1993); L'Huillier et al., EMBO J. 11:4411-8 (1992); Lisziewicz et al., Proc. Natl. Acad. Sci. U.S.A 90:8000-4 (1993); Thompson et al., Nucleic Acids Res. 23:2259 (1995); Sullenger & Cech, Science 262:1566 (1993)).

### Host Cells and Methods of Recombinantly Producing Protein of the Invention

Nucleic acid molecules encoding growth factor, colony stimulating factor or M-CSF polypeptides, or growth factor, colony stimulating factor or M-CSF fusion proteins of this invention and vectors comprising these nucleic acid molecules can be used for transformation of a suitable host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

Transformation of appropriate cell hosts with a rDNA molecule of the present invention is accomplished by well known methods that typically depend on the type of vector used and host system employed. With regard to transformation of prokaryotic host cells, electroporation and salt treatment methods can be employed (see, for example, Sambrook et al., Molecular Cloning—A Laboratory Manual, Cold Spring Harbor Laboratory Press (1989); Cohen et al., Proc. Natl. Acad. Sci. USA 69:2110-2114 (1972)). M-CSF has been successfully purified from the prokarytoic host cells, E. coli, in monomer form and renatured to generate fully active dimers. (Halenbeck et al., Biotechnology 7:710-15 (1889)) With regard to transformation of vertebrate cells with vectors containing rDNA, electroporation, cationic lipid or salt treatment methods can be employed (see, for example, Graham et al., Virology 52:456-467 (1973); Wigler et al., Proc. Natl. Acad. Sci. USA 76:1373-1376 (1979)).

Successfully transformed cells, i.e., cells that contain a rDNA molecule encoding a growth factor, colony stimulating factor or M-CSF, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern, J. Mol. Biol. 98:503-517 (1975) or the proteins produced from the cell may be assayed by an immunological method.

Host cells for expression of a polypeptide or antibody of the invention for use in a method of the invention may be prokaryotic or eukaryotic. Mammalian cell lines available as hosts for expression are well known in the art and include many immortalized cell lines available from the American Type Culture Collection (ATCC®). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines. Cell lines of particular preference are selected through determining which cell lines have high expression levels. Other useful eukaryotic host cells include plant cells. Other cell lines that may be used are insect cell lines, such as Sf9 cells. Exemplary prokaryotic host cells are E. coli and Streptomyces.

When recombinant expression vectors encoding the growth factor, colony stimulating factor or M-CSF polypeptides and growth factor, colony stimulating factor or M-CSF proteins of the invention are introduced into mammalian host cells, they are produced by culturing the host cells for a period of time sufficient to allow for expression of the antibody, polypeptide and fusion polypeptide in the host cells or, more preferably, secretion of the growth factor, colony stimulating factor or M-CSF polypeptides and growth factor, colony stimulating factor or M-CSF fusion proteins of the invention into the culture medium in which the host cells are grown. Growth factor, colony stimulating factor or M-CSF polypeptides and growth factor, colony stimulating factor or M-CSF fusion proteins of the invention can be recovered from the culture medium using standard protein purification methods.

Further, expression of growth factor, colony stimulating factor or M-CSF polypeptides and growth factor, colony stimulating factor or M-CSF fusion proteins of the invention of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

A polypeptide produced by a cultured cell as described herein can be recovered from the culture medium as a secreted polypeptide, or, if it is not secreted by the cells, it can be recovered from host cell lysates. As a first step in isolating the polypeptide, the culture medium or lysate is generally centrifuged to remove particulate cell debris. The polypeptide thereafter is isolated, and preferably purified, from contaminating soluble proteins and other cellular components, with the following procedures being exemplary of suitable purification procedures: fractionation on immunoaffinity or ion-exchange columns; ethanol precipitation; reverse phase HPLC; chromatography on silica or on a cation-exchange resin such as DEAE; chromatofocusing; SDS PAGE; ammonium sulfate precipitation; and gel filtration, e.g., with Sephadex™ columns (Amersham Biosciences). Protease inhibitors may be used to inhibit proteolytic degradation during purification. One skilled in the art will appreciate that purification methods suitable for the polypeptide of interest may require modification to account for changes in the character of the polypeptide upon expression in recombinant cell culture.

The purification of polypeptides may require the use of, e.g., affinity chromatography, conventional ion exchange chromatography, sizing chromatography, hydrophobic interaction chromatography, reverse phase chromatography, gel filtration or other conventional protein purification techniques. See, e.g., Deutscher, ed. (1990) “Guide to Protein Purification” in Methods in Enzymology, Vol. 182.

### Compositions

The growth factor polypeptides, colony stimulating factor polypeptides, M-CSF polypeptides, polypeptide fragments, polynucleotides, vectors and host cells of the invention may be formulated into pharmaceutical compositions for administration to animals, including humans. In some embodiments, the invention provides compositions comprising a growth factor, colony stimulating factor or M-CSF polypeptide or fusion protein of the present invention.

In some embodiments, the invention provides compositions comprising a growth factor polypeptide or fusion protein. In some embodiments, the invention provides a composition comprising a colony stimulating factor polypeptide or fusion protein. In some embodiments, the invention provides a composition comprising an M-CSF polypeptide or fusion protein

In some embodiments, the invention provides compositions comprising a growth factor polynucleotide. In some embodiments, the invention provides a composition comprising an colony stimulating factor polynucleotide. In some embodiments, the invention provides a composition comprising an M-CSF polynucleotide.

In some embodiments, the compositions may contain a monomer of dimer of MCSF. The dimer can be a homodimer composed of polypeptides having the above recited M-CSF reference amino acid sequences or a heterodimer composed of any two polypeptides having the above recited M-CSF reference amino acid sequences. The polypetides present in the homo- or heterodimer may be substituted M-CSF polypeptides or polypeptide fragments having at least 70%, 75%, 80%, 85%, 90%, or 95% identical to polypeptides of SEQ ID NOS:1, 2 or 3 or fragments thereof.

In some embodiments, the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension including, for example, sodium carboxymethyl cellulose, sorbitol and dextran. Optionally, the suspension may also contain stabilizers. Liposomes can also be used to encapsulate the molecules of this invention for delivery into the cell. Exemplary “pharmaceutically acceptable carriers” are any and all solvents, dispersion media, coatings, antibacterial and antifungal agents, isotonic and absorption delaying agents, and the like that are physiologically compatible, water, saline, phosphate buffered saline, dextrose, glycerol, ethanol and the like, as well as combinations thereof. In some embodiments, the composition comprises isotonic agents, for example, sugars, polyalcohols such as mannitol, sorbitol, or sodium chloride. In some embodiments, the compositions comprise pharmaceutically acceptable substances such as wetting or minor amounts of auxiliary substances such as wetting or emulsifying agents, preservatives or buffers, which enhance the shelf life or effectiveness of the soluble Nogo receptors or fusion proteins of the invention.

Compositions of the invention may be in a variety of forms, including, for example, liquid, semi-solid and solid dosage forms, such as liquid solutions (e.g., injectable and infusible solutions), dispersions or suspensions. The preferred form depends on the intended mode of administration and therapeutic application. In one embodiment, compositions are in the form of injectable or infusible solutions, such as compositions similar to those used for passive immunization of humans with other antibodies.

The composition can be formulated as a solution, micro emulsion, dispersion, liposome, or other ordered structure suitable to high drug concentration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In some embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See e.g., Sustained and Controlled Release Drug Delivery Systems, J. R. Robinson, ed., Marcel Dekker, Inc., New York (1978).

The pharmaceutical compositions of the invention may include a “therapeutically effective amount” or a “prophylactically effective amount” of a polypeptide(s), or fusion protein of the invention. A “therapeutically effective amount” refers to an amount effective, at dosages and for periods of time necessary, to achieve the desired therapeutic result. A therapeutically effective amount of the growth factor polypeptide, growth factor protein, colony stimulating factor polypeptide, colony stimulating factor protein, M-CSF polypeptide or M-CSF protein may vary according to factors such as the disease state, age, sex, and weight of the individual. A therapeutically effective amount is also one in which any toxic or detrimental effects of the growth factor polypeptide, growth factor protein, colony stimulating factor polypeptide, colony stimulating factor protein, M-CSF polypeptide or M-CSF protein are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The composition may be administered as a single dose, multiple doses or over an established period of time in an infusion. Dosage regimens also may be adjusted to provide the optimum desired response (e.g., a therapeutic or prophylactic response). For example, the growth factor, colony stimulating factor or M-CSF may be administered to an animal once per day, one week out of the month, continuously (e.g., by osmotic pump), intermittently, before, during or after formation of amyloid deposits or plaques, until there is a reduction in the size and/or formation of amyloid deposits or plaques or a combination of two or more thereof. In addition, over time, the dose may be proportionally reduced or increased as indicated by the exigencies of the therapeutic situation. It is especially advantageous to formulate parenteral compositions in dosage unit form for ease of administration and uniformity of dosage unit form as used herein refers to physically discrete units suited as unitary dosages for the mammalian subjects to be treated, each unit containing a predetermined quantity of active compound calculated to produce the desired therapeutic effect in association with the required pharmaceutical carrier. The specification for the dosage unit forms of the invention are dictated by and directly dependent on (a) the unique characteristics of the growth factor polypeptide, growth factor protein, colony stimulating factor polypeptide, colony stimulating factor protein, M-CSF polypeptide or M-CSF protein and the particular therapeutic or prophylactic effect to be achieved, and (b) the limitations inherent in the art of compounding such a growth factor polypeptide, growth factor protein, colony stimulating factor polypeptide, colony stimulating factor protein, M-CSF polypeptide or M-CSF protein for the treatment of sensitivity in individuals. In some embodiments a therapeutically effective dose range for the growth factor, colony stimulating factor or M-CSF polypeptide is 0.001-10 mg/Kg per day. In some embodiments a therapeutically effective dose range for the growth factor, colony stimulating factor or M-CSF polypeptides thereof is 0.01-1 mg/Kg per day. In some embodiments a therapeutically effective dose range for the growth factor, colony stimulating factor or M-CSF polypeptides thereof is 0.05-0.5 mg/Kg per day. In some embodiments a therapeutically effective dose range for the growth factor, colony stimulating factor or M-CSF polypeptides thereof is 0.05-0.2 mg/Kg per day. In some embodiments a therapeutically effective dose range for the growth factor, colony stimulating factor or M-CSF polypeptides thereof is 0.001-0.5 mg/Kg per day.

For treatment with a growth factor, colony stimulating factor or M-CSF polypeptide, the dosage can range, e.g., from about 0.0001 to 100 mg/kg, from about 0.001 to 0.5 mg/kg, or 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly.

In some methods, two or more growth factors, colony stimulating factors or M-CSF polypeptides or fusion proteins are administered simultaneously, in which case the dosage of each polypeptide or fusion protein administered falls within the ranges indicated. Supplementary active compounds also can be incorporated into the compositions used in the methods of the invention. For example, a growth factor, colony stimulating factor or M-CSF polypeptide or fusion protein may be coformulated with and/or coadministered with one or more additional therapeutic agents, such as an adrenergic, anti-adrenergic, anti-androgen, anti-anginal, anti-anxiety, anticonvulsant, antidepressant, anti-epileptic, antihyperlipidemic, antihyperlipoproteinemic, antihypertensive, anti-inflammatory, antiobessional, antiparkinsonian, antipsychotic, adrenocortical steroid; adrenocortical suppressant; aldosterone antagonist; amino acid; anabolic steroid; analeptic; androgen; blood glucose regulator; cardioprotectant; cardiovascular; cholinergic agonist or antagonist; cholinesterase deactivator or inhibitor, cognition adjuvant or enhancer; dopaminergic; enzyme inhibitor, estrogen, free oxygen radical scavenger; GABA agonist; glutamate antagonist; hormone; hypocholesterolemic; hypolipidemic; hypotensive; immunizing; immunostimulant; monoamine oxidase inhibitor, neuroprotective; NMDA antagonist; AMPA antagonist, competitive or -non-competitive NMDA antagonist; opioid antagonist; potassium channel opener; non-hormonal sterol derivative; post-stroke and post-head trauma treatment; prostaglandin; psychotropic; relaxant; sedative; sedative-hypnotic; selective adenosine antagonist; serotonin antagonist; serotonin inhibitor; selective serotonin uptake inhibitor; serotonin receptor antagonist; sodium and calcium channel blocker; steroid; stimulant; and thyroid hormone and inhibitor agents.

In some embodiments, the growth factor, colony stimulating factor or M-CSF polypeptide or fusion protein is administered by a route selected from the group consisting of oral administration; nasal administration; parenteral administration; transdermal administration; topical administration; intraocular administration; intrabronchial administration; intraperitoneal administration; intravenous administration; subcutaneous administration; intramuscular administration; and a combination of two or more of these routes of administration.

Parenteral injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference in its entirety.

The invention encompasses any suitable delivery method for a growth factor, colony stimulating factor or M-CSF polypeptide or fusion protein to a selected target tissue, including bolus injection of an aqueous solution or implantation of a controlled-release system. Use of a controlled-release implant reduces the need for repeat injections.

The compositions may also comprise a growth factor, colony stimulating factor or M-CSF polypeptide or fusion protein of the invention dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers 22:547-56 (1985)); poly (2-hydroxyethyl-methacrylate), ethylene vinyl acetate (Langer et al., J. Biomed. Mater. Res. 15:167-277 (1981); Langer, Chem. Tech. 12:98-105 (1982)) or poly-D-(−)-3hydroxybutyric acid (EP 133,988).

Supplementary active compounds also can be incorporated into the compositions used in the methods of the invention. For example, a growth factor, colony stimulating factor or M-CSF polypeptide or fragment thereof, or a fusion protein thereof, may be coformulated with and/or coadministered with one or more additional therapeutic agents. For example, a growth factor, colony stimulating factor or M-CSF polypeptide or polypeptide fragment, or a fusion protein thereof, may be coformulated with and/or coadministered with a therapeutic agent effective to treat, ameliorate or prevent AD, including but not limited to cholinesterase inhibitors, such as galantamine, rivastigmine, tacrine and donepezil; and N-methyl D-aspartate (NMDA) antagonists, such as memantine.

Suitable additional agents that may be may be coformulated with and/or coadministered with a growth factor, colony stimulating factor or M-CSF polypeptide or polypeptide fragment are described elsewhere herein.

For treatment with a growth factor, colony stimulating factor or M-CSF polypeptide or polypeptide fragment of the invention, the dosage can range, e.g., from about 50 to about 100 μg/kg body weight per day of the host body weight, from about 1 to about 500 μg/kg body weight per day of the host body weight or from about 50 to about 200 μg/kg body weight per day. Doses intermediate in the above ranges (e.g., 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 pg/kg, 70 μg/kg, 75 μg/kg, 80 μg/kg, 85 μg/kg, 90 μg/kg, 95 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg) are also intended to be within the scope of the invention. Subjects can be administered such doses daily, on alternative days, weekly or according to any other schedule determined by empirical analysis. In one embodiment, treatment entails administration in multiple dosages over a prolonged period, for example, of at least six months. In another embodiment, treatment regimes entail administration once per week, once every two weeks, once per month or once every 3 to 6 months.

In addition to systemic administration, the growth factors, colony stimulating factors or M-CSF polypeptides and polypeptide fragments used in the methods of the invention may be concurrently directly infused into an organ or tissue, such as the brain.

The growth factors, colony stimulating factors or M-CSF polypeptides and polypeptide fragments used in the methods of the invention may be concurrently directly infused into the brain. Various implants for direct brain infusion of compounds are known and are effective in the delivery of therapeutic compounds to human patients suffering from neurological disorders. These include chronic infusion into the brain using a pump, stereotactically implanted, temporary interstitial catheters, permanent intracranial catheter implants, and surgically implanted biodegradable implants. See, e.g., Gill et al., supra; Scharfen et al., Int. J. Radiation Oncology Biol. Phys. 24(4):583-91 (1992); Gaspar et al., Int. J. Radiation Oncology Biol. Phys. 43(5):977-82 (1999); chapter 66, pages 577-580, Bellezza et al., “Stereotactic Interstitial Brachytherapy,” in Gildenberg et al., Textbook of Stereotactic and Functional Neurosurgery, McGraw-Hill (1998); and Brem et al., J. Neuro-Oncology 26:111-23 (1995).

In some embodiments, the method of treatment of the invention involves concurrent administration of a growth factor, colony stimulating factor or M-CSF polypeptide or polypeptide fragment to an animal by direct infusion into an appropriate region of an organ or tissue, such as the brain. See, e.g., Gill et al., Nature Med. 9: 589-95 (2003). Alternative techniques are available and may be applied to administer an M-CSF polypeptide according to the invention. For example, stereotactic placement of a catheter or implant can be accomplished using the Riechert-Mundinger unit and the ZD (Zamorano-Dujovny) multipurpose localizing unit. A contrast-enhanced computerized tomography (CT) scan, injecting 120 ml of omnipaque, 350 mg iodine/ml, with 2 mm slice thickness can allow three-dimensional multiplanar treatment planning (STP, Fischer, Freiburg, Germany). This equipment permits planning on the basis of magnetic resonance imaging studies, merging the CT and MRI target information for clear target confirmation.

The Leksell stereotactic system (Downs Surgical, Inc., Decatur, Ga.) modified for use with a GE CT scanner (General Electric Company, Milwaukee, Wis.) as well as the Brown-Roberts-Wells (BRW) stereotactic system (Radionics, Burlington, Mass.) can be used for this purpose. Thus, on the morning of the implant, the annular base ring of the BRW stereotactic frame can be attached to the patient's skull. Serial CT sections can be obtained at 3 mm intervals though the (target tissue) region with a graphite rod localizer frame clamped to the base plate. A computerized treatment planning program can be run on a VAX 11/780 computer (Digital Equipment Corporation, Maynard, Mass.) using CT coordinates of the graphite rod images to map between CT space and BRW space.

The compositions used in the method of the invention may also comprise a growth factor, colony stimulating factor or M-CSF polypeptide or polypeptide fragment of the invention dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers 22:547-56 (1985)); poly(2-hydroxyethyl-methacrylate), ethylene vinyl acetate (Langer et al., J. Biomed. Mater. Res. 15:167-277 (1981); Langer, Chem. Tech. 12:98-105 (1982)) or poly-D-(−)-3hydroxybutyric acid (EP 133,988)

### Cell Therapy

In some embodiments of the invention a soluble growth factor, colony stimulating factor or M-CSF polypeptide is administered in a treatment method that includes: (1) transforming or transfecting an implantable host cell with a nucleic acid, e.g., a vector, that expresses a soluble growth factor, colony stimulating factor or M-CSF polypeptide; and (2) implanting the transformed host cell into a mammal, at the site of a disease, disorder or injury. For example, the transformed host cell can be implanted at the site of a spinal cord injury. In some embodiments of the invention, the implantable host cell is removed from a mammal, temporarily cultured, transformed or transfected with an isolated nucleic acid encoding a soluble growth factor, colony stimulating factor or M-CSF polypeptide, and implanted back into the same mammal from which it was removed. The cell can be, but is not required to be, removed from the same site at which it is implanted. Such embodiments, sometimes known as ex vivo gene therapy, can provide a continuous supply of the soluble growth factor, colony stimulating factor or M-CSF polypeptide, localized at the site of site of action, for a limited period of time.

### Gene Therapy

A soluble growth factor, colony stimulating factor or M-CSF polypeptide can be produced in vivo in a mammal, e.g., a human patient, using a gene-therapy approach to treatment of a disease, disorder or injury in which reducing amyloid accumulation would be therapeutically beneficial. This involves administration of a suitable soluble growth factor, colony stimulating factor or M-CSF polypeptide-encoding nucleic acid operably linked to suitable expression control sequences. Generally, these sequences are incorporated into a viral vector. Suitable viral vectors for such gene therapy include an adenoviral vector, an alphavirus vector, an enterovirus vector, a pestivirus vector, a lentiviral vector, a baculoviral vector, a herpesvirus vector, an Epstein Barr viral vector, a papovaviral vector, a poxvirus vector, a vaccinia viral vector, adeno-associated viral vector and a herpes simplex viral vector. The viral vector can be a replication-defective viral vector. Adenoviral vectors that have a deletion in its El gene or E3 gene are typically used. When an adenoviral vector is used, the vector usually does not have a selectable marker gene. Examples of such vectors can be found in PCT publications WO 2006/060089 and WO2002/056918 which are incorporated herein in their entireties.

Expression constructs of a growth factor, colony stimulating factor or soluble M-CSF polypeptide may be administered in any biologically effective carrier, e.g. any formulation or composition capable of effectively delivering the soluble growth factor, colony stimulating factor or M-CSF polypeptide gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g. a cDNA, encoding a soluble growth factor, colony stimulating factor or M-CSF polypeptide, or a soluble growth factor, colony stimulating factor or M-CSF polypeptide antisense nucleic acid. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes. A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include .psi.Crip, .psi.Cre, .psi.2 and .psi.Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Reviewed in Ali, 2004, Novartis Found Symp. 255:165-78; and Lu, 2004, Stem Cells Dev. 13(1):133-45. Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol. 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of a soluble growth factor, colony stimulating factor or M-CSF polypeptide, fragment, or analog, in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject growth factor, colony stimulating factor or M-CSF gene by the targeted cell. Exemplary gene delivery systems of this type include liposomal derived systems, poly-lysine conjugates, and artificial viral envelopes. Other embodiments include plasmid injection systems such as are described in Meuli et al. (2001) J Invest Dermatol. 116(1):131-135; Cohen et al. (2000) Gene Ther 7(22): 1896-905; or Tam et al. (2000) Gene Ther 7(21):1867-74.

In a representative embodiment, a gene encoding a soluble growth factor, colony stimulating factor or M-CSF polypeptide, active fragment, or analog, can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075).

In clinical settings, the gene delivery systems for the therapeutic growth factor, colony stimulating factor or M-CSF gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) Pros. Natl. Acad. Sci. USA 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

Guidance regarding gene therapy in particular for treating a CNS condition or disorder as described herein can be found, e.g., in U.S. patent application Ser. No. 2002/0,193,335 (provides methods of delivering a gene therapy vector, or transformed cell, to neurological tissue); U.S. patent application Ser. No. 2002/0,187,951 (provides methods for treating a neurodegenerative disease using a lentiviral vector to a target cell in the brain or nervous system of a mammal); U.S. patent application Ser. No. 2002/0,107,213 (discloses a gene therapy vehicle and methods for its use in the treatment and prevention of neurodegenerative disease); U.S. patent application Ser. No. 2003/0,099,671 (discloses a mutated rabies virus suitable for delivering a gene to the CNS); and U.S. Pat. No. 6,436,708 (discloses a gene delivery system which results in long-term expression throughout the brain); U.S. Pat. No. 6,140,111 (discloses retroviral vectors suitable for human gene therapy in the treatment of a variety of disease); and Kaspar et al. (2002) Mol. Ther. 5:50-6′ Suhr et al (1999) Arch Neurol. 56:287-92; and Wong et al. (2002) Nat Neurosci 5, 633-639).

Production of Recombinant Proteins Using a rDNA Molecule

The present invention further provides methods for producing a growth factor, colony stimulating factor or M-CSF polypeptide and/or a growth factor, colony stimulating factor or M-CSF fusion protein of the invention using nucleic acid molecules herein described. In general terms, the production of a recombinant form of a protein typically involves the following steps: First, a nucleic acid molecule is obtained that encodes a protein of the invention. If the encoding sequence is uninterrupted by introns, it is directly suitable for expression in any host. The nucleic acid molecule is then optionally placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

### Methods Using Growth Factor Polypeptides, Fusion Proteins, Polynucleotides and Compositions

One embodiment of the present invention provides a method for increasing the activity of bone marrow-derived microglial cells in an organ or tissue of a mammal, e.g., the brain, comprising systemically administering an effective amount of a growth factor polypeptide, for example, a colony stimulating factor, for example, M-CSF, GM-CSF, or G-CSF.

One embodiment of the present invention provides a method for increasing the activity of bone marrow-derived microglial cells in the brain of a mammal, comprising systemically administering an effective amount of growth factor polypeptide, for example, a colony stimulating factor, for example, M-CSF, GM-CSF, or G-CSF.

Another embodiment of the invention provides a method for reducing amyloid plaques in an organ or tissue of a mammal, comprising systemically administering an amount of growth factor polypeptide, for example, a colony stimulating factor, for example, M-CSF, GM-CSF, or G-CSF effective to increase the activity of bone marrow-derived microglial cells in an organ or tissue of that mammal.

Another embodiment of the invention provides a method for reducing Aβ plaques in the brain of a mammal, comprising systemically administering an amount of growth factor polypeptide, for example, a colony stimulating factor, for example, M-CSF, GM-CSF, or G-CSF effective to increase the activity of bone marrow-derived microglial cells in the brain of that mammal.

Another embodiment of the invention provides a method of reducing the number of amyloid plaques in an organ or tissue of a mammal, comprising systemically administering an amount of growth factor polypeptide, for example, a colony stimulating factor, for example, M-CSF, GM-CSF, or G-CSF effective to increase the activity of bone marrow-derived microglial cells in an organ or tissue of that mammal.

Another embodiment of the invention provides a method of reducing the number of Aβ plaques in the brain of a mammal, comprising systemically administering an amount of growth factor polypeptide, for example, a colony stimulating factor, for example, M-CSF, GM-CSF, or G-CSF effective to increase the activity of bone marrow-derived microglial cells in the brain of that mammal.

Another embodiment of the invention provides a method of reducing the size of amyloid plaques in an organ or tissue of a mammal, comprising systemically administering an amount of growth factor polypeptide, for example, a colony stimulating factor, for example, M-CSF, GM-CSF, or G-CSF effective to increase the activity of bone marrow-derived microglial cells in an organ or tissue of that mammal.

Another embodiment of the invention provides a method of reducing the size of Aβ plaques in the brain of a mammal, comprising systemically administering an amount of growth factor polypeptide, for example, a colony stimulating factor, for example, M-CSF, GM-CSF, or G-CSF effective to increase the activity of bone marrow-derived microglial cells in the brain of that mammal.

A further embodiment of the invention provides a method for improving memory function or inhibiting memory loss in a mammal comprising systemically administering an amount of growth factor polypeptide, for example, a colony stimulating factor, for example, M-CSF, GM-CSF, or G-CSF effective to increase the activity of bone marrow-derived microglial cells in the brain of that mammal.

An additional embodiment of the invention provides a method for treating disorders associated with amyloid plaques, for e.g., amyloidosis, comprising systemically administering an amount of growth factor polypeptide, for example, a colony stimulating factor, for example, M-CSF, GM-CSF, or G-CSF effective to increase the activity of bone marrow-derived microglial cells in an organ or tissue of that mammal.

An additional embodiment of the invention provides a method for treating disorders associated with β-amyloid plaques, including AD, comprising systemically administering an amount of growth factor polypeptide, for example, a colony stimulating factor, for example, M-CSF, GM-CSF, or G-CSF effective to increase the activity of bone marrow-derived microglial cells in the brain of that mammal.

Disease that can be treated using the methods of the present invention include but are not limited to AD, mild cognitive impairment, mild-to-moderate cognitive impairment, vascular dementia, cerebral amyloid angiopathy, hereditary cerebral hemorrhage, senile dementia, Down's syndrome, inclusion body myositis, age-related macular degeneration, multiple myeloma, pulmonary hypertension, congestive heart failure, cerebral amyloid angiopathy (CAA), type II diabetes, rheumatoid arthritis, familial amyloid polyneuropathy (FAP), spongiform encephlaopathies, Parkinson's disease, primary systemic amylodoisis, secondary systemic amyloidosis, fronto-temporal dementias, senile systemic amyloidosis, hereditary cerebral amyloid angiopathy, haemodialysis-related amyloidosis, familial amyloid polyneuropathy III, Finnish hereditary systemic amyloidosis, medullary carcinoma of the thyroid, atrial amyloidosis, hereditary non-neuropathic systemic amyloidosis, injection-localized amyloidosis, hereditary renal amyloidosis, amyotrophic lateral sclerosis, Huntington's disease, spinal and bulbar muscular atrophy, spinocerebellar ataxias and spinocerebellar ataxia 17, inclusion myositis or a condition associated with AD. Conditions associated with AD that can be treated using the methods of the present invention include but are not limited to hypothyroidism, cerebrovascular disease, cardiovascular disease, memory loss, anxiety, a behavioral dysfunction, a neurological condition, or a psychological condition. Behavioral dysfunction that can be treated using the methods of the present invention include but is not limited to apathy, aggression, or incontinence. Neurological conditions that can be treated using the methods of the present invention include but are not limited to Huntington's disease, amyotrophic lateral sclerosis, acquired immunodeficiency, Parkinson's disease, aphasia, apraxia, agnosia, Pick disease, dementia with Lewy bodies, altered muscle tone, seizures, sensory loss, visual field deficits, in coordination, gait disturbance, transient ischemic attack or stroke, transient alertness, attention deficit, frequent falls, syncope, neuroleptic sensitivity, normal pressure hydrocephalus, subdural hematoma, brain tumor, posttraumatic brain injury, or posthypoxic damage. Psychological conditions that can be treated using the methods of the present invention include but are not limited to depression, delusions, illusions, hallucinations, sexual disorders, weight loss, psychosis, a sleep disturbance, insomnia, behavioral disinhibition, poor insight, suicidal ideation, depressed mood, irritability, anhedonia, social withdrawal, or excessive guilt.

Mild cognitive impairment (MCI) is a condition characterized by a state of mild but measurable impairment in thinking skills, but is not necessarily associated with the presence of dementia. MCI frequently, but not necessarily, precedes AD. It is a diagnosis that has most often been associated with mild memory problems, but it can also be characterized by mild impairments in other thinking skills, such as language or planning skills. However, in general, an individual with MCI will have more significant memory lapses than would be expected for someone of their age or educational background. As the condition progresses, a physician may change the diagnosis to mild-to-moderate cognition impairment, as is well understood in this art.

In methods of the present invention, a growth factor polypeptide, for example, a colony stimulating factor, for example, M-CSF, is systemically administered. “Systemically administered” includes any route of administration except for intercranially.

The growth factor, colony stimulating factor or M-CSF polypeptides or fusion proteins of the present invention can be provided alone, or in combination, or in sequential combination with other agents that modulate a particular pathological process. As used herein, the growth factor, colony stimulating factor or M-CSF and growth factor, colony stimulating factor or M-CSF receptor fusion proteins, are said to be administered in combination with one or more additional therapeutic agents when the two are administered simultaneously, consecutively or independently.

In some embodiments, a growth factor, colony stimulating factor or M-CSF polypeptide or fusion protein may be coformulated with and/or coadministered with one or more anti-amyloid antibodies, e.g., anti-β-amyloid antibody, for use in the methods of the present invention. Examples of anti-Aβ for use in the methods of the present invention can be found, e.g., in U.S. Patent Publication Nos. 20060165682 A1, 20060039906 A1, and 20040043418 A1.

In some embodiments, a growth factor, colony stimulating factor or M-CSF polypeptide or fusion protein may be coformulated with and/or coadministered with one or more additional therapeutic agents, such as an adrenergic agent, anti-adrenergic agent, anti-androgen agent, anti-anginal agent, anti-anxiety agent, anticonvulsant agent, antidepressant agent, anti-epileptic agent, antihyperlipidemic agent, antihyperlipoproteinemic agent, antihypertensive agent, anti-inflammatory agent, antiobessional agent, antiparkinsonian agent, antipsychotic agent, adrenocortical steroid; adrenocortical suppressant; aldosterone antagonist; amino acid; anabolic steroid; analeptic agent; androgen; blood glucose regulator; cardioprotectant agent; cardiovascular agent; cholinergic agonist or antagonist; cholinesterase deactivator or inhibitor, cognition adjuvant or enhancer; dopaminergic agent; enzyme inhibitor, estrogen, free oxygen radical scavenger; GABA agonist; glutamate antagonist; hormone; hypocholesterolemic agent; hypolipidemic agent; hypotensive agent; immunizing agent; immunostimulant agent; monoamine oxidase inhibitor, neuroprotective agent; NMDA antagonist; AMPA antagonist, competitive or -non-competitive NMDA antagonist; opioid antagonist; potassium channel opener; non-hormonal sterol derivative; post-stroke and post-head trauma treatment; prostaglandin agent; psychotropic agent; relaxant agent; sedative agent; sedative-hypnotic agent; selective adenosine antagonist; serotonin antagonist; serotonin inhibitor; selective serotonin uptake inhibitor; serotonin receptor antagonist; sodium and calcium channel blocker; steroid; stimulant; melphalan followed by autologous stem cell transplantation to support bone marrow recovery (HDM/SCT); colchicine; metal chalators; small molecule inhibitors of amyloid formations; benzothiazoles; 4′-dianilino-1,1′binaphthyl-5,5′-sulfonate (bis-ANS); phthalocyanine tetrasulfonate; and thyroid hormone and inhibitor agents for use in the methods of the present invention.

The dosage administered will be dependent upon the age, health, and weight of the recipient, kind of concurrent treatment, if any, frequency of treatment, and the nature of the effect desired. The compounds of this invention can be utilized in vivo, ordinarily in mammals, such as humans, sheep, horses, cattle, pigs, dogs, cats, rats and mice, or in vitro.

The methods of treatment of diseases and disorders as described herein are typically tested in vitro, and then in vivo in an acceptable animal model, for the desired therapeutic or prophylactic activity, prior to use in humans. Suitable animal models, including transgenic animals, are will known to those of ordinary skill in the art. The effect of the growth factor polypeptides, fusion proteins, and compositions on increasing the activity of bone marrow-derived microglial cells in an organ or tissue, for e.g., the brain, can be tested in vitro as described in the Examples. Finally, in vivo tests can be performed by creating transgenic mice which express the appropriate phenotype and administering the growth factor, colony stimulating factor or M-CSF polypeptides to mice or rats in models as described herein.

It will be readily apparent to one of ordinary skill in the relevant arts that other suitable modifications and adaptations to the methods and applications described herein are obvious and may be made without departing from the scope of the invention or any embodiment thereof. In order that this invention may be better understood, the following examples are set forth. These examples are for purposes of illustration only and are not to be construed as limiting the scope of the invention in any manner.

### EXAMPLES

### Example 1

### Production and Purification of M-CSF

Construction of an Exemplary Mammalian Expression Vector p3ACSF-69

To construct a mammalian vector for expression of an M-CSF protein, the cDNA sequence of SEQ ID NO: 1 can be adapted with restriction endonuclease enzyme XhoI linkers (New England Biolabs) and ligated into XhoI-digested, phosphatased COS cell expression vector pXM. pXM contains the SV40 enhancer, adenovirus major late promoter, DHFR coding sequence, SV40 late message poly A addition site and VaI gene. pXM further contains a linker sequence with restriction endonuclease sites for KpnI, PstI and XhoI. The plasmid resulting from the XhoI digestion of pXM and the insertion of the linker and the XhoI adapted DNA sequence of SEQ ID NO: 1 coding for a M-CSF protein can then be transformed by conventional techniques into a suitable mammalian host cell for expression of the M-CSF protein. Exemplary host cells are mammalian cells and cell lines, particularly primate cell lines, rodent cell lines and the like.

A similar expression vector may also be prepared containing the other, M-CSF sequences identified above (SEQ ID NOS: 2 and 3), or containing only the amino acid coding regions of those sequences with the 5′ and 3′ non-coding regions deleted. One skilled in the art can construct other mammalian expression vectors comparable to that described above by cutting the DNA sequence of SEQ ID NO: 1 from the plasmid with XhoI and employing well-known recombinant genetic engineering techniques and other known vectors, such as pCD [Okayama et al., Mol. Cell. Biol. 2:161-170 (1982)] and pJL3, pJL4 [Gough et al., EMBO J. 4:645-653 (1985)]. The transformation of these vectors into appropriate host cells can result in expression of an M-CSF protein.

Similarly, one skilled in the art could manipulate the M-CSF sequences by eliminating or replacing the mammalian regulatory sequences flanking the coding sequence with yeast, bacterial or insect sequences to create non-mammalian vectors expressable in yeast, bacterial or insect host cells. For example, the coding sequence of SEQ ID NO: 1 could be cut from the mammalian vector construct described above with XhoI and further manipulated (e.g., ligated to other known linkers or modified by deleting non-coding sequences therefrom or altering nucleotides therein by other known techniques). The modified M-CSF coding sequence could then be inserted into, for example, a known bacterial vector using procedures such as described in T. Taniguchi et al., Proc. Natl. Acad. Sci. U.S.A., 77:5230-5233 (1980). This exemplary bacterial vector could then be transformed into bacterial host cells and the M-CSF protein expressed thereby.

Similar manipulations can be performed for the construction of an insect vector [See, e.g., procedures described in published European patent application 155,476] or a yeast vector [See, e.g., procedures described in published PCT application WO 86 00639] for expression of the M-CSF proteins in insect or yeast cells.

### Example 2

### Expression of an M-CSF Protein

Plasmid DNA, prepared from E. coli HB101 containing the mammalian expression vector described above, as described in Maniatis et al., Molecular Cloning, A Laboratory Manual, Cold Spring Harbor Laboratory, (1982) can be purified by conventional methods involving equilibrium centrifugation in cesium chloride gradients containing ethidium bromide. COS cells (ATCC CRL 1650) are transfected with the purified DNA at a concentration of approximately 5 μg plasmid DNA per 10⁶ COS cells and treated with chloroquine according to the procedures described in G. G. Wong et al., Science, 280:810-815 (1985) and R. J. Kaufman et al., Mol. Cell. Biol., 2:1304 (1982). 72 hours following transfection, the medium can be harvested and will contain a protein which demonstrates M-CSF activity in standard bone marrow assays.

One method for producing high levels of M-CSF from mammalian cells involves the construction of cells containing multiple copies of the heterologous M-CSF gene. The heterologous gene can be linked to an amplifiable marker, e.g., the dihydrofolate reductase (DHFR) gene for which cells containing increased gene copies can be selected for by propagation in increasing concentrations of methotrexate (MTX) according to the procedures of Kaufman & Sharp, J. Mol. Biol., 159: 601-629 (1982). This approach can be employed with a number of different cell types.

The mammalian expression vector described above contains the M-CSF gene in operative association with other plasmid sequences enabling expression thereof. The M-CSF mammalian expression vector and the DHFR expression plasmid pAdA26SV(A)3 (Kaufman & Sharp, Mol. Cell. Biol., 2:1304 (1982)) can be co-introduced into DHFR-deficient CHO cells, DUKX-BII, by calcium phosphate coprecipitation and transfection. DHFR expressing transformants are selected for growth in alpha media with dialyzed fetal calf serum, and subsequently selected for amplification by growth in increasing concentrations of MTX (sequential steps in 0.02, 0.2, 1.0 and 5 μM MTX) as described in Kaufman, et al., Mol. Cell. Biol. 5: 1750 (1983). Transformants are cloned, and biologically active M-CSF protein expression is monitored by murine bone marrow assays. M-CSF expression should increase with increasing levels of MTX resistance.

### Example 3

M-CSF Affects on Bone Marrow-Derived Microglial Cell Activity and on β-Amyloid Plaques in the Brain

To examine whether systemically administered M-CSF increases the activity of bone marrow-derived microglial cells in the brain of a mouse, the presence of bone marrow-derived microglial cells in brain lysates is assayed. In addition, to see whether an increase in the activity of bone marrow-derived microglial cells in the brain of a mouse reduces β-amyloid plaques in the brain, the presence and size of β-amyloid plaques is assayed.

Mice are divided into several treatment groups. The first group serves as a normal control group. Each additional group is treated with isolated M-CSF or an active fragment variant or derivative thereof by systemic administration. The amount administered varies for each treatment group. For example, groups may receive 1, 10, 25, 50, 75, 100, 200, 300, 400, or 500 μg/kg per day, respectively. Administration can be a one time administration or can occur daily for a specified period of time, e.g., 3, 5, 7, 10, 14 or 20 days. Mice are sacrificed between 3 and 14 days after administration.

Mice are sacrificed following anaesthetization via an intraperitoneal injection of sodium pentobarbital (0.5 mg/g body weight). Their brains are removed and fixed in 4% paraformaldehyde, followed by rinsing in 0.1 M of phosphate-buffered saline (PBS). Specimens are then embedded in paraffin and 10 μm frontal serial sections are cut. Sections are incubated for 1 h with F4/800 antiserum (Secrotec, Oxford, UK) diluted 1:1000 in PBS. Rabbit polyclonal antiserum raised against F4/80 glycoprotein purified from mouse macrophages is antigen specific for monocytes and mature macrophages. After washing in PBS-Triton, biotinylated goat anti-rabbit serum (Vector Laboratories, Burlingame, Calif., USA) diluted 1:200 in PBS is applied for 45 minutes. The sections are washed, incubated in alcoholic 0.3% hydrogen peroxide for 20 minutes to quench endogenous peroxidase activity, washed again and then incubated with the avidin-biotin complex (Vectostatin Elite ABC kit, Vector Laboratories) for 45 minutes. Control sections, for example, were normal rabbit serum replaces F4/80 antiserum at the same dilution can be routinely processed alongside test sections. The peroxidase is visualized using 0.05% diaminobenzidine hydrochloride (DAB; Sigma Chemical Co., St. Louis, Mo., USA), 0.08% imidazole, and 0.05% hydrogen peroxide. To increase the contrast for the morphometric study, the DAB reaction product can be intensified using 0.01% osmium tetroxide. Cell nuclei are counterstained with methyl green. Adjacent sections are stained with cresyl violet acetate to identify the boundaries of the structures that are examined.

Sections are stained immunohistochemically with a primary antibody against Aβ for 12 h at 4° C. using Vectastain Elite ABC kits. In addition, to confirm Aβ deposition, consecutive sections can be analyzed by Congo red staining.

The number of fibrillar plaques and microglial cells are calculated in the cerebral cortex, hippocampus, amygdala and hypothalamus from three sections taken from around Bregma −3.14 mm using the atlas of Paxinos and Watson. Paxinos and Watson eds, The Rat Brain in Sterotaxic Coordinates, 4th ed. NY Acad. Press (1998).

### Example 4

### The Interaction Between Bone-Marrow Derived-Microglial Cells and β Amyloid Plaques

The following experiments provide a means to determine the interaction between bone-marrow derived-microglial cells and β amyloid plaques.

### Preparation of Cy3-Labeled Amyloid-β

β-amyloid1-42 (Anaspec, San Jose, Calif.) is dissolved at 1 mg/ml in 50 mM phosphate buffer (pH 7.0-7.3) and conjugated with Cy3 monofunctional dye (Amersham Bioscience, Piscataway, N.J.) following the manufacturer's guidelines. Briefly, 5 μl of coupling buffer is thoroughly mixed to 100 μl of the solution of amyloid peptides, the resulting solution is then mixed to the vial of Cy3 reactive dye and incubated 30 min at room temperature with additional mixing every 10 min. Unconjugated dye is separated from the labeled peptides by dialysis overnight in a 3.5 K MWCO Slide-A-Lyzer dialysis cassette (Pierce, Rockford, Ill.). Fluorescent intensity of dialyzed Cy3-labeled amyloid peptides is measured with a SLM AMINCO Bowman AB2 spectrofluorimeter (Exc: 550±4 nm; Em: 564±4 nm; sensitivity: 835 volts, high-voltage enable) and compared to a nondialysed one (100% of Cy3 dye. The solution of Cy3-labeled amyloid peptide is used later as a fluorescent tracer when mixed with nonlabeled amyloid proteins in cell culture medium.

### Cell Culture and Immunocytochemistry

BV2 microglial cells are routinely grown in DMEM (Gibco, Invitrogen, Burlington, ON) supplemented with 10% fetal bovine serum (Wisent, St-Bruno, QC), 100 units of penicillin/ml, 100 μg of streptomycin/ml, at pH 7.4 and 37° C. in an H₂O-saturated, 5% CO₂ atmosphere. Cells are seed at 10000 cells per well in height-chamber glass slides (Lab-Tek, Nalge Nunc International, Rochester, N.Y.). Two days later, cells are incubated for 1 hr with 25 μg/ml b-amyloid1-42 (Anaspec, San Jose, Calif.) and 2.5 μg/ml of Cy3-labeled b-amyloid1-42. Thereafter, cells are rinsed several times with HBSS and then fixed with 4% formaldehyde (pH 7.4) during 15 min at 37° C. Cells are rinsed three times with HBSS. Chambers are removed from the slide, and cells are coverslipped with a polyvinyl alcohol (Sigma-Aldrich) mounting medium containing 2.5% 1,4-diazabicyclo(2,2,2)-octane (Sigma-Aldrich) in buffered glycerol (Sigma-Aldrich).

### Confocal Laser Scanning Microscopy for Phagocytosis Experiments

Confocal laser scanning microscopy is performed with a BX-61 microscope equipped with the Fluoview SV500 imaging software 4.3 (Olympus America Inc, Melville, N.Y.), using a 100× Plan-Apochromat oil-immersion objective (NA 1.35) and a 2-3.5× zoom ratio in the region of interest. Cy3-labeled amyloid peptide is excited at 543 nm using an argon-He laser (MellesGriot Laser Group [Carlsbad, Calif.]) set at 70% of maximum power. Fluorescence emission from Cy3 dye is recorded by photomultipliers with emission filter preset within FV500 software (Red pseudocolor; BA: 560-600 nm). Transmission channel is captured in the same time to delineate the shape of the cells. 0.1 μm confocal z-series are acquired for each observation area and filtered by three frame Kalman low speed scans. Acquired z-series images are exported in Imaris Pro Sofware 4.2.0 (Bitplane AG, Zurich, CH).

### Tridimensional Reconstructions, Modelings, and Animations

Z-series of the different experiments are imported from the Olympus Fluoview format to the Imaris Pro Software running on a Dell Precision 650 dual Intel Xeon workstation equipped with 4 GB of RAM and a PNY Quadro FX3000G graphic accelerator. Image thresholding and channel pseudocolors are adjusted, and 3D reconstruction is performed in a Surpass Scene as follows: orthogonal view of either the maximum intensity projection (MIP) or the blend projection of the volume of the stacked images are captured, first, channel by channel, and then all the channels together in the Surpass module. Pictures are cropped with Adobe Photoshop CS and thereafter assembled in Adobe Illustrator CS. Modeling of the objects is performed in perspective view, channel by channel; while overlaying carefully with 3D rotations the objects from the original MIP volume with the new isosurfaces generated by advanced Gaussian filter/Threshold level settings. Objects are automatically closed at the border. Light source is set to optimize the 3D rendering effects on the textures wrapping the different objects. Animations are created in two steps. First, movements of the amyloid plaque in 3D are added as key frames in the animation scenario. In a second step, several effects, such as MIP background for volume/blend background for models, zoom in/out, opacity/transparency of specific channels, yellow selection boxes, clipping planes, and ortho slicers are added at specific time points to look inside and around the amyloid plaque and put in evidence the intimate relationships between the plaque and the surrounding microglial cells. High-resolution movies are exported in the .avi file format and then heavily compressed with Microsoft Windows Movie Maker.

### Example 5

Reduction of Aβ Plaque Load in M-CSF treated APPswe/PSEN-14E9 mice

Treatment of APPswe/PS-1ΔE9 transgenic mice is initiated at 7 months of age when the mice have become symptomatic, as judged by Aβ deposition in brain and by reduced spatial memory function (see below). After 3 months of systemic treatment M-CSF, the brain is examined by immunohistochemistry and ELISA. Aβplaques in parasagittal sections are fixed by paraformaldehyde and labeled with anti-Aβ-(1-17) 6E10 antibody after 0.1 M formic acid treatment. Plaque area is quantitated using NIH Image as a percentage of total cerebral cortical area for two sections from each animal. Data are ±SEM.

### Example 6

### Radial Arm Water Maze Performance in APPswe/PSEN-1ΔE9 Transgenic Mice

The ability of systemic administration of M-CSF to reduce Aβ plaque is encouraging, but cognitive performance is the relevant symptom in clinical AD. APPswe/PS-1ΔE9 (Park et al., J Neurosci 26:1386-1395 (2006)) are obtained from Jackson Laboratories (Bar Harbor, Me.) (Stock #04462). To assess APPswe/PS-1AE9 transgene-related impairments in spatial memory, a modified radial arm water maze paradigm (RAWM) is employed. Morgan et al., Nature 408:982-985 (2000). A modified radial arm water maze testing protocol is based on personal communication with D. Morgan (Morgan et al., Nature 408:982-985 (2000)). The maze consists of a circular pool one meter in diameter with six swim alleys nineteen cm wide that radiates out from a 40 cm open central area and a submerged escape platform is located at the end of one arm. Spatial cues are presented on the walls and at the end of each arm. The behaviorist is blind to treatment. To control for vision, motivation and swimming, mice are tested in an open water visual platform paradigm for up to one minute and latency times are recorded. Next, mice are placed in a random arm according to an Excel function =MOD($CELL+RANDBETWEEN(1,5),6), where $CELL is the location of the hidden platform. Each mouse is allowed to swim up to one minute to find the escape platform. Upon entering an incorrect arm (all four paws within that swim alley) or failing to select an arm after twenty seconds, the mouse is pulled back to the start arm and charged an error. All mice spend 30 seconds on the platform following each trial before beginning the next trial. Thereafter, the mouse is tested four more times, constituting a learning block. Mice are allowed to rest for 30 minutes between learning blocks. In total, mice are tested over three learning blocks over the first day and on the following day another three learning blocks are repeated.

Differences in the number of swim errors between treated and untreated mice indicate an improvement in memory function.

### Example 7

### Macrophage Colony-Stimulating Factor (M-CSF) Regulation of Macrophage and Monocyte Production in Mice

Macrophage colony-stimulating factor (M-CSF) is a hematopoietic growth factor that stimulates the proliferation of monocyte/macrophage precursors, and the differentiation of these precursor cells into mature macrophages. The effects of this compound are tested in a model of neurodegeneration. For this model, 128 male mice of strain C57/B16 and 12-24 weeks in age are used. Doses and dosing paradigms of a recombinant human M-CSF on a hematological endpoint are first evaluated. Evaluation and validation of the use of complete blood cell counts, cytochemical differentials and flow cytometry as pharmacodynamic endpoints for these studies are then performed. Evaluation and comparison of the use of a subcutaneous or intraperitoneal pump for continuous dosing as compared to daily subcutaneous, intravenous or intraperitoneal injections are also performed.

In general, animals are treated with a medium dose of M-CSF daily for days. Peripheral monocytosis is used as a pharmacological marker. Peak monocytosis typically occurs on days 5-7. Animals are bled then euthanized on the final day of treatment. Blood is analyzed via the hematology machine and FACS, the spleen is removed weighed and then flash frozen for potential histology.

Dosing Administration and Regimens

Using a subcutaneous or intraperitoneal pump or daily intravenous, intraperitoneal or subcutaneous bolus injections (as determined by data from experiment 1 a dose response curve is generated to determine a dose for additional studies. Three M-CSF concentrations are studied and one control group. Treatment is administered for 4 weeks. Blood is sampled in a staggered method across groups to capture changes in blood cell counts on a twice a week basis without allowing for hemodynamic alterations due directly to changes in blood volume (blood is drawn from individual animals every 2 weeks).

Dosing regimens for these experiments include the following:

| Agent(s) | Dose/Volume | Route | Frequency Duration |
|---|---|---|---|
| M-CSF | 100 μg/kg/day, >0.2 mls/20 g | SC | pump/daily, 5 days |
| M-CSF | 30-500 μg/kg/day, 0.25 μl/hr | SC | pump/daily, 4 wks |

Animals receive daily subcutaneous injections of vehicle or drug, or receive subcutaneous pumps which deliver the same daily concentration of drug. Implantation of subcutaneous osmotic pumps is a surgical procedure and is outlined below. ALZET pumps operate because of an osmotic pressure difference between a compartment within the pump, called the salt sleeve, and the tissue environment in which the pump is implanted. The high osmolality of the salt sleeve causes water to flux into the pump through a semipermeable membrane which forms the outer surface of the pump. As the water enters the salt sleeve, it compresses the flexible reservoir, displacing the test solution from the pump at a controlled, predetermined rate. Because the compressed reservoir cannot be refilled, the pumps are designed for single-use only. In the case of the 4 week study, pumps will be replaced at least 1 time (a 20 gram mouse can hold a 200 microliter pump which will run at this rate for 2 weeks).

Animals are anesthetized with isoflurane until it no longer responds to a toe pinch. Once the animal is anesthetized, the skin over the implantation site is shaved and washed using betadine scrub and 70% isopropanol (alternating 3×). A mid-scapular incision approximately 3 mm long is made and a hemostat is inserted into the incision, and, by opening and closing the jaws of the hemostat, spread the subcutaneous tissue to create a pocket for the pump. A filled pump is inserted into the pocket, delivery portal first. The wound is then closed with wound clips or sutures. On day 14, animals are prepared as stated above, an incision is made, the pump is removed and another pump is placed as stated above.

Pumps are inserted in mice to allow for chronic subcutaneous injects. The size of the mice limits the use of pumps to one that contains a volume of 200 microliters and is replaced after 14 days.

Addition models with higher pump rates are also used. 200 microliter and 100 microliter pumps are appropriate for subcutaneous implantation in a 20 gram mouse. Pumps with rates that range from 0.25 microliters/hour to those that reach 1 microliter/hour to achieve the concentrations are used. Weekly or biweekly exchange of the osmotic pumps is determined following the initial experiment based on the final dose range and size of the animals. Pump size and rate is selected to allow for the proper dosing regimen while minimizing the amount of surgery the animals undergo (i.e. smaller pumps are used in a 5 day study and larger pumps are selected for a 30 day study).

In addition, larger animals and an increased pump speed are used, for example, if the 500 micrograms/kg dose is deemed necessary following the original experiment (determining that pumps and subcutaneous daily injections are equal and changes in Monocyte production can be determined using CBC and Facs).

Experiment 1

16 mice are given a medium range subcutaneous dose of M-CSF daily for 5 days (peak monocytosis typically occurred on days 5-7) (8 with pumps and 8 with daily injections), while 16 mice are given the vehicle as a control (8 via pump and 8 via daily injections). Eight animals are needed in a group for statistical significance, for validating the CBC machine versus FACS.

| | | |
|---|---|---|
| Treatment | Daily Injections | = 8 |
| | mp | = 8 |
| Vehicle | Daily Injections | = 8 |
| | mp | = 8 |

N = 32

Experiment 2

Four groups of animals are given either one of 3 doses of M-CSF or vehicle, for 4 weeks. Each group contains 12 animals, to allow for alternate bleeding schedules and permit complete recovery between cycles and still capture the full range of changes induced by the M-CSF.

| Low Dose | N = 12 |
|---|---|
| Middle Dose | =12 |
| High Dose | =12 |
| Vehicle | =12 |

N = 48 × 2 = 96

As those skilled in the art will appreciate, numerous changes and modifications may be made to the preferred embodiments of the invention without departing from the spirit of the invention. It is intended that all such variations fall within the scope of the invention.

### Example 8

### Macrophage Colony-Stimulating Factor (M-CSF) APP Mice Morris Water Maze Analysis

Four groups of 6 month old mice (19 APP and 23 APP″) were injected intraperitoneal (I.P.) 3 times a week with either M-CSF or vehicle (PBS) for 10 weeks. A day after treatment ended, mice were tested in a Morris Water Maze (MWM). (Morris, J. Neurosci Methods 11(1):47-60 (9184)).

The Morris water maze task was conducted in a black circular pool of a diameter of 100 cm. Tap water was filled in with a temperature of 22±1° C., cold enough to give the mice an additional motivation to escape from the water. The pool was virtually divided into four quadrants. A transparent platform (6 cm diameter) was placed about 1 cm beneath the water surface, so it offered no local cues to guide escape behavior. During the whole test session, except the pretest, the platform was located in the southwest quadrant of the pool.

MWM testing consisted of three stages:

Firstly, because this test presupposes seeing abilities, we exclusively investigated black-eyed animals (which rarely have visual problems compared to albinos). In addition, animals performed a pretest allowing judging whether seeing abilities are normal. During this test, animals had two changes (two trials) to swim to the visible platform marked with a familiar object or a marker of some sort, e.g., a black and white pole. In each turn a mouse performed three swimming trials on four consecutive days. A single trial lasts for a maximum of one minute.

Secondly, in hidden platform training, 3-Dimensional visual cues were added to the walls of the facility, and the black and white pole removed. The mice were placed into the tank and swam around the tank to find the hidden platform (up to 90 seconds). If the animal does not find the “way” out of the water, the investigator guides to or places the mouse on the platform. After each trial mice were allowed to rest on the platform for 10-15 sec. During this time, the mice have the possibility to orientate in the surrounding. Investigations took place under dimmed light conditions, to prevent negative influences on the tracking system (Kaminski; PCS, Biomedical Research Systems). On the walls around the pool, posters with black, bold geometric symbols (e.g. a circle and a square) were fixed. Despite the challenging light situation, the mice used these symbols as landmarks for their orientation. This was the acquisition phase. Each mouse swam 4 times a day for 6 days.

Thirdly, following hidden platform training, mice were placed in the tank with no platform, and allowed to swim freely for 40 seconds. The percentage time spent on the target quadrant was calculated as a measure of spatial reference memory. To control for vision, motivation and swimming, mice are tested in an open water visual platform paradigm for up to one minute and latency times are recorded. For the quantification of escape latency (the time [second]—the mouse needs to find the hidden platform to escape from the water), pathway (the length [meter] to reach the target) and abidance in the goal quadrant a computerized tracking system was used. The computer was connected to a camera placed above the centre of the pool. The camera detected the signal of the light emitting diode (LED) fixed with a little hairgrip on the mouse's tail.

Two mice were excluded from the analysis due to high levels of thigmotaxis and floating. These mice were not included in the final statistical analysis.

Results.

There was no difference in latency between any of the groups in the visible platform stage of the MWM, indicating that mice in all groups had comparable motivation and ability to find the platform (no visual, motivational and swimming deficiencies among groups) (FIG. 1).

The observed data also suggested that there was no difference in latency between any of the groups on the first or second days of hidden platform trials, showing that all of the groups found the task equally challenging during the first two days (FIG. 1A). Overall, mice in the four different groups (APP⁺, M-CSF, APP⁻, MSCF, APP⁺, PBS, APP⁻, PBS) performed equally well when presented with a visual platform in Morris water maze, demonstrating that mice in all groups had similar levels of motivation, response to visual cues, and motor skills (reflected in swimming proficiency). In the first 2 days of hidden platform test mice from different groups also performed at comparable levels, demonstrating that all groups initially found the test equally challenging.

On day 3 of hidden platform trials, APP⁺, PBS mice performed worse than APP⁺, M-CSF mice (Students't-test, p=0.02); APT, M-CSF mice (Students' t-test, p=0.02); and APT, PBS, APP⁺ mice (Students' t-test, p=0.02) (FIG. 1).

On day 4 of hidden platform trials, APP⁺, PBS mice performed significantly worse than APT, M-CSF mice (p=0.01), and APT, PBS mice (p=0.03) (FIG. 1).

On day 5 of hidden platform trials, APP⁺PBS mice performed significantly worse than APT, M-CSF mice (p=0.02) (FIG. 1).

On day 6 of hidden platform trials, APP⁺, PBS mice performed significantly worse than APT, M-CSF mice (p=0.02) (FIG. 1).

Overall, the difference in latency of the 4 groups of mice over 6 days of hidden platform testing compared by a repeated measures ANOVA was highly statistically significant (p=0.009) (FIG. 1). The key comparison of APP⁺, PBS mice and APP⁺, M-CSF mice by a repeated measures ANOVA was also statistically significant (p=0.02) (FIG. 1).

Therefore, it was concluded that, MSCF treatment returned the impaired ability of APP⁺ mice to acquire spatial memory to wild-type levels.

1. A method of increasing tissue resident macrophage activity in an organ or tissue of an animal afflicted with an amyloidosis, comprising systemically administering to an animal in need thereof a composition comprising an active ingredient selected from the group consisting of:
(a) an isolated colony stimulating factor polypeptide or active variant, fragment or derivative thereof;
(b) an isolated polynucleotide encoding a colony stimulating factor polypeptide or active variant, fragment or derivative thereof and
(c) a combination of (a) and (b), and
wherein said composition is administered in an amount effective to increase tissue resident macrophage activity in said animal, thereby treating said amyloidosis.

2. The method of claim 1, wherein said increase in tissue resident macrophage activity in said organ or tissue is effected by an increase in the number of tissue resident macrophages in said organ or tissue.

3. The method of claim 1, wherein said increase in tissue resident macrophage activity in said organ or tissue is effected by an increase in the functional activity of tissue resident macrophages in said organ or tissue.

4. The method of claim 1, wherein said increase in tissue resident macrophage activity in said organ or tissue is effected by an increase in the targeting of tissue resident macrophages in said organs or tissues.

5. The method of claim 1, wherein said tissue resident macrophage is microglia.

6. The method of claim 5, wherein said microglia is bone marrow-derived microglia.

7. (canceled)

8. The method of claim 1, wherein the amyloidosis comprises the formation of amyloid plaques or amyloid protein aggregates that are phagocytosed by said tissue resident macrophages.

9. (canceled)

10. The method of claim 1, wherein said amyloid plaques or aggregates are reduced in number, reduced in size or a combination thereof.

11. The method of claim 1, wherein the amyloidosis comprises the formation of amyloid plaques or amyloid protein aggregates that comprise a protein selected from a group consisting of β-amyloid, immunoglobulin light chain, serum amyloid A, β₂-microglobulin, drusen, wild-type or mutant transthyretin, mutant apolipoprotein AI, mutant apolipoprotein AII, islet amyloid precursor protein, calcitonin, atrial natriuretic protein, huntingtin, human prion protein in “scrapie” form, α-synuclein, tau, cystatin C, gelsolin, amylin, mutant lysozyme, insulin, superoxide dismutase I, androgen receptor, ataxins, TATA box-binding protein, mutant fibrinogen A α-chain, β-protein precursor, and a combination of amyloid proteins.

12. The method of claim 1, wherein said amyloidosis is selected from the group consisting of Alzheimer's disease, mild cognitive impairment, mild-to-moderate cognitive impairment, vascular dementia, cerebral amyloid angiopathy (CAA), senile dementia, trisomy 21 (Down's syndrome), hereditary cerebral hemorrhage with amyloidosis of the Dutch-type (HCHWA-D), inclusion body myositis, age-related macular degeneration, multiple myeloma, pulmonary hypertension, congestive heart failure, type II diabetes, rheumatoid arthritis, familial amyloid polyneuropathy (FAP), spongiform encephlaopathies, Parkinson's disease, primary systemic amylodoisis, secondary systemic amyloidosis, fronto-temporal dementias, senile systemic amyloidosis, hereditary cerebral amyloid angiopathy, haemodialysis-related amyloidosis, familial amyloid polyneuropathy III, Finnish hereditary systemic amyloidosis, medullary carcinoma of the thyroid, atrial amyloidosis, hereditary non-neuropathic systemic amyloidosis, injection-localized amyloidosis, hereditary renal amyloidosis, amyotrophic lateral sclerosis, Huntington's disease, spinal and bulbar muscular atrophy, spinocerebellar ataxia, inclusion myocytis and combinations thereof.

13. The method of claim 12, wherein said amyloidosis is Alzheimer's disease.

14-15. (canceled)

16. The method of claim 1, wherein the colony stimulating factor is selected from the group consisting of macrophage colony stimulating factor, granulocyte colony stimulating factor, granulocyte-macrophage colony stimulating factor, an active variant, fragment or derivative of any said colony stimulating factors, and a combination of two or more of said colony stimulating factors or active variants, fragments or derivatives thereof.

17. The method of claim 16, wherein the colony stimulating factor is macrophage colony stimulating factor (M-CSF) or an active variant, fragment or derivative thereof.

18-34. (canceled)

35. The method of claim 1, wherein said composition comprises is an isolated colony stimulating factor polypeptide or active variant, fragment or derivative thereof.

36. The method of claim 17, wherein said composition comprises a homodimer comprising two identical active M-CSF polypeptide fragments.

37. The method of claim 17, wherein said composition comprises a heterodimer comprising two different active M-CSF polypeptide fragments.

38. The method of claim 1, wherein said composition comprises an isolated polynucleotide encoding a colony stimulating factor polypeptide or active variant, fragment or derivative thereof, through operable association with a promoter, and wherein said polynucleotide is delivered via an expression vector.

39-44. (canceled)

45. The method of claim 35, wherein said colony stimulating factor or active variant, fragment or derivative thereof further comprises a second polypeptide fused thereto and wherein the second polypeptide is selected from the group consisting of an immunoglobulin Fc region, a serum albumin moiety, a targeting moiety, a reporter moiety, a purification-facilitating moiety, and a combination of two or more thereof.

46-49. (canceled)

50. The method of claim 1, wherein said effective amount of colony stimulating factor or active fragment, variant or derivative thereof is between about 50 and about 100 μg/kg per day.

51-53. (canceled)

54. The method of claim 1, further comprising administering a stem cell factor or active variant, fragment or derivative thereof or a pharmaceutical compound effective for treating, preventing or inhibiting amyloidosis.

55-61. (canceled)
